(19) United States
(12) Patent Application Publication
(10) Pub. No.: US 2004/0127447 A1
(43) Pub. Date: Jul. 1, 2004

(54) Augmentation of K+ channel expression using adenoviral vectors

(76) Inventors: Stephen L. Archer, Edmonton (CA); Evangelos D. Michelakis, Edmonton (CA)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/671,253

(22) Filed: Sep. 25, 2003

Related U.S. Application Data
(60) Provisional application No. 60/413,911, filed on Sep. 27, 2002.

(51) Int. Cl. A61K 48/00; C12N 15/00

(52) U.S. Cl. 514/44; 435/320.1

(57) ABSTRACT

The invention provides a recombinant adenoviral vector comprising nucleic acid encoding at least one K+ channel gene operably linked to a promoter for the augmentation of K+ channel expression and the treatment of vascular diseases.

### FIELD OF THE INVENTION

The present invention relates to the augmentation of K+ channel expression using adenoviral vectors and more particularly to gene therapy for pulmonary hypertension, patent ductus arteriosus and other vascular diseases.

### BACKGROUND OF THE INVENTION

Exposure to chronic hypoxia reliably leads to the development of chronic hypoxic pulmonary hypertension (CH-PHT). CH-PHT is a model for the PHT that occurs in patients with chronic lung diseases or people residing at high altitude. Hypoxic pulmonary vasoconstriction (HPV) is selectively blunted in rats with CH-PHT, whilst response to other vasoconstrictors is preserved or enhanced, as discussed in McMurtry I F, et al. (1978) Am J Physiol. 235:H104-H109; and Isaacson T C, et al. (1994) J. Appl. Physiol. 76:933-940.

The loss of O₂ sensitivity and development of PHT are associated with decreased expression of O₂ and 4-aminopyridine (4-AP)-sensitive K+ channels (Kv1.5 and Kv2.1), both in cultured pulmonary artery smooth muscle cells (PASMC), as discussed in Wang J, et al. (1997) J. Clin. Invest. 100:2347-2353, and intact Pas, as discussed in Reeve H L, et al. (2001) J Appl Physiol. 90:2249-56.

HPV is a vasomotor response that occurs in the pulmonary circulation in response to alveolar hypoxia. HPV occurs in small resistance pulmonary arteries (PA) and serves to constrict arteries supplying hypoxic alveoli. This improves ventilation to perfusion matching in the lung and thereby optimizes systemic oxygenation. HPV is intrinsic to the PA, occurring ex vivo in isolated, perfused lungs and PA rings. In addition, HPV has also been demonstrated in isolated PASMCs, suggesting that the fundamental mechanism, though modulated importantly by many endothelial mediators, is intrinsic to the PASMC. In the pulmonary circulation, Kv channels, including α-subunits of Kv1.5 and Kv2.1, are responsible for determining the membrane potential of PASMCs. Inhibition of PASMC Kv channels with 4-AP results in pulmonary vasoconstriction. As K⁺ current (I_{K}) is inhibited, the plasmalemmal membrane potential (E_{M}) depolarizes, thereby increasing the opening of L-type voltage-dependent Ca⁺⁺ channels, increasing inward Ca⁺⁺ current and activating the contractile apparatus, causing vasoconstriction. Likewise, hypoxia inhibits I_{K} and depolarizes rat PASMCs. Candidate O₂-sensitive Kv channels that have been implicated in this response are Kv1.5 and Kv2.1/9.3, as discussed in Patel A J, et al., (1997) Embo J. 16:6615-25 and Archer S L, et al. (1998) J Clin Invest. 101:2319-30, in addition to Kv1.2 and Kv3.1b, as discussed in Hulme J T, et al. (1999) Circ Res. 85:489-97, and Osipenko O N, et al., (2000) Circ Res. 86:534-40.

Pharmacologic evidence has recently been provided to show that O₂-induced constriction of the human ductus arteriosus (DA) also involves inhibition of voltage-gated potassium channels (Kv) in ductus arteriosus smooth muscle cell (DASMC), as discussed in Michelakis E, et al. (2000) Lancet 356:134-7.

In the fetus, the ductus arteriosus (DA) is tonically relaxed by its hypoxic environment. This allows venous blood to bypass the nonventilated lungs. At birth, simultaneous pulmonary vasodilatation and DA vasoconstriction direct right ventricular blood flow into the pulmonary circulation. Functional closure of the DA (due to vasoconstriction) precedes anatomical closure (due to cell proliferation) by days and is crucial to the newborn's transition to an air-breathing organism. The DA constrictor response to O₂, though modulated by the endothelium (reinforced by endothelin, ET, and inhibited by vasodilatory prostanoids and nitric oxide, NO), is intrinsic to the DA smooth muscle cell (DASMC). O₂ constriction persists ex vivo, following endothelial-denudation, and despite inhibition of prostaglandin H synthase (PGHS), nitric oxide synthase (NOS) and ET-A receptors. As in most arteries, this K⁺ channel inhibition leads to SMC depolarization, opening of the voltage-gated L-type Ca⁺⁺ channels, influx of Ca⁺⁺ and vasoconstriction. However, the molecular identity of the relevant DASMC Kv channels, as well as the mechanism of O₂ sensing in this important human vessel, remain unknown.

Vascular O₂ sensing systems consist of a sensor, the function of which is altered by changes in PO₂, a mediator, produced by this sensor, and an effector, which alters vascular tone in response to the mediator. Many aspects of the O₂ sensor-effector pathway are conserved amongst O₂-sensitive mammalian tissues, the pulmonary artery (PA), the DA, the adrenomedulllary cells, the neuroepithelial body and the carotid body. In each tissue, K⁺ channels have been implicated in the effector mechanism, as discussed in Archer S L, et al., (2000) Adv Exp Med Biol. 475:219-40, whilst the O₂ sensor has been proposed to involve a change in redox state, as determined by mitochondria or NADPH oxidase.

Attention has focused on the mitochondria because electron transport chain (ETC) inhibitors mimic hypoxia, constricting the PA and activating the carotid body. Mitochondria respond to changes in PO₂ by altering their respiration and production of reactive O₂ species (ROS). This changes cellular redox potential and alters the function of many redox-sensitive genes, second messenger systems and O₂ -sensitive K⁺ channels in the membrane, prior to any depletion of ATP. Because K⁺ channels control SMC membrane potential (Em), and thus tone, in most vascular beds, O₂ sensitive K⁺ channels are attractive candidate effectors in vascular redox-based O₂ sensing systems. O₂-sensitive K⁺ channels include homo- and heterotetramers composed of the several Kv α-subunits (Kv1.2, Kv1.5, Kv2.1, Kv3.1b, Kv4.3, Kv9.3) and the calcium-sensitive K⁺ channels, BK_{Ca}, as discussed in Hulme Jtet al., (1999) Circ Res.;85:489-97; Archer S L, et al., (1998) J Clin Invest. 101:2319-30; Patel A J, et al., (1997) EMBO J. 16:6615-25; Perez-Garcia M T, et al., (2000) J Neurosci. 20:5689-95; Cornfield D N, et al., (1996) Proc. Natl. Acad. Sci. 93:8089-8094; and Riesco-Fagundo A M, et al., (2001) Circ Res. 89:430-6. In rabbit DAs, O₂ , and oxidants such as H₂O₂, inhibit DASMC Kv current and depolarize E_{M}, leading to vasoconstriction.

Previous studies suggest that decreased expression and activity of Kv channels is causally related to the loss of HPV and the development of CH-PHT. Previous studies also suggest that O₂ constricts the human DA via inhibition of DASMC Kv channels. The present invention relates to the restoration of Kv channels, using selective gene transfer accomplished by nebulization of an adenoviral vector, for reducing CH-PHT and restoring HPV.

All publications cited herein are hereby incorporated by reference in their entirety.

### SUMMARY OF THE INVENTION

In a broad aspect the present invention provides an expression vector, preferably a recombinant adenoviral vector, comprising nucleic acid encoding at least one K+ channel gene, operably linked to a promoter. In an alternative embodiment of the invention the vector further comprises a reporter gene operably linked to the nucleic acid.

The present invention further provides a method of treating a vascular disease comprising administering to a person having a vascular disease a composition comprising an expression vector, preferably a recombinant adenoviral vector, encoding at least one K+ channel gene operably linked to a promoter, and a pharmaceutically acceptable excipient. In an alternative embodiment of the invention the vector further comprises a reporter gene operably linked to the nucleic acid. This vector may be administered by, for example, nebulization of the airway, by intravascular routes or by direct injection (or topically).

### BRIEF DESCRIPTION OF THE DRAWINGS

The invention will be better understood with reference to the attached detailed description and to the following Figures, wherein:

FIG. 1 illustrates the effect of gene transfer on pulmonary vascular resistance index (PVRI), cardiac index (CI), pulmonary artery pressure (PAP) and right ventricular/left ventricular plus septum measurements;

FIG. 2A illustrates representative traces to show that the normal magnitude of HPV is blunted in the CH-PHT;

FIG. 2B shows the effect of gene transfer on HPV and AII;

FIG. 3A illustrates a representative confocal microscopy images showing green fluorescence in a lung section from a CH-Ad5-Kv1.5 rat and a CH-S rat;

FIGS. 3B and C are immunoblots on lung tissue for GFP, Kv1.5, and Kv2.1;

FIG. 4 is an immunohistochemistry for human Kv1.5 and illustrates Kv1.5 overexpressed in CH-Ad5-Kv1.5 Pas;

FIGS. 5 and 6 illustrate the effect of gene transfer on K⁺ Current density;

FIGS. 7A-E illustrate Kv channels control of DA tone;

FIG. 8 illustrates mRNA<Kᵥ1.5 and Kᵥ2.1 expressed in the media and SMCs of DA;

FIGS. 9A-F illustrates the parallel effects of ETC-inhibitors and hypoxia on tone and I_{K} in human DA;

FIGS. 10A and B illustrate the exposure of DA to chronic normoxia causes loss of sensitivity of tone and Iₖ to both O₂ and 4-AP;

FIG. 11A shows the confocal microscopy of DA rings;

FIG. 11B shows the confocal microscopy for DASMCs grown in normoxic versus hypoxic primary culture;

FIGS. 11C and D illustrate E_{M} measured in green fluorescent units in chronically normoxic DASMCs and chronic hypoxic controls;

FIG. 11E illustrates the results of qRT-PCR for Kv1.5 and Kv2.1 mRNA in tissue cultured rings;

FIG. 12A shows ex vivo gene transfer in intact DA rings confirmed by confocal microcopy;

FIG. 12B illustrates the Kv current in DASMCs from the infected DA;

FIG. 12C illustrates the results of qRT-PCR specific for the expression of rat Kv2.1 in Das;

FIGS. 12D and E illustrates that Kv2.1 gene transfer significantly restores the responsiveness of the chronically normoxic DA ring to both 4-AP and O₂;

FIGS. 13A and B are images of human DASMC showing an elaborate mitochondrial network (100X);

FIG. 13C illustrates the effects of antimycin, rotenone and cyanide on TMRM-loaded DASMC mitochondria;

FIGS. 14A and B illustrate the effects on Lucigenin-enhanced chemiluminescence and H₂O₂ production when PO₂ levels are increased in isolated human DA rings;

FIGS. 14C and D illustrate the effects of t-butyl-H₂O₂ on DASMCs;

FIG. 14E illustrates the proposed mechanism for O₂-constriction in the DA;

FIG. 15 illustrates the calibration curve for the AmplexRed assay;

FIG. 16 is an immunohistochemistry showing the presence of Kv2.1 and Kv1.5 α-subunit proteins in DASMCs;

FIG. 17 illustrates the RT-PCR was performed on individual ducti;

FIG. 18 illustrates the expression of subunits of ETC complexes I-IV in DA rings; and

FIG. 19 is shows the TMRM images of the mitochondria of a HL-1 cardiac myocyte cell line and DASMCs.

### DETAILED DESCRIPTION OF THE INVENTION

The present invention discloses the administration of Kv channel genes, and more preferably Kv1.5, to the pulmonary circulation via an aerosol, and effectiveness of the administration in eliciting transgene expression in resistance PASMCs. The administration of Kv1.5, cloned from human PA, reduces PVRI in experimental PHT. Kv gene therapy with an O₂-sensitive channel restores HPV and O₂ sensitive I_{K} in rats with established CH-PHT. Examples of the Kᵥ channel genes that may be administered include, but are not limited to, Kv1.5, Kv2.1/9.3, large conductance calcium-sensitive K+ channel, Kv1.2, Kv3.1 and BK_{Ca}.

The present invention further discloses the administration of Kv intravascularly, and more preferably Kv2.1, preferably by catheter, for modulation of tone in the patent ductus arteriosus.

The present invention further discloses an expression vector comprising nucleic acid encoding at least one K+ channel gene operably linked to a promoter. The present invention further discloses the vector having a reporter gene operably linked to the nucleic acid. The vector can be used in the administration of Kv to the pulmonary circulation. Preferably the expression vector is an adenoviral vector, and more preferably the expression vector is a replication-deficient adenoviral vector. However, it will be understood by a person skilled in the art that any vector system may be used in the treatment of vascular disease that has a suitable efficiency for gene transfer and for long term expression, examples include, but are not limited to, the gutless adenovirus, lenti virus, naked DNA, nonviral vectors, adenoassociated virus, cationic polymers of the polylysine, polyethlenimine, an DEAE-neutral lipids, lipofectants, cationic lipids such as lipopolyamines and the herpes virus.

Preferably the promoter that is used is a tissue specific promoter. Examples of promoters that may be used include, but are not limited to, the CMV promoter and the SM-22 promoter. It will be understood by a person skilled in the art that any suitable reporter gene may be used. Examples include, but are not limited to, the green fluorescent protein gene (GFP) and the myc tag.

The present invention further provides an isolated host cell stably transformed with an expression vector comprising nucleic acid encoding at least one K+ channel gene operably linked to a promoter.

The present invention further provides an article of manufacture comprising packaging material within which is contained a nebulizable formulation useful to treat a vascular disease, wherein the formulation comprises an expression vector comprising nucleic acid encoding at least one K+ channel gene operably linked to a promoter with a pharmaceutically excipient, and the packaging material comprises a label which indicates that the formulation can be used to treat a vascular disease.

The present invention is directed to the central role for Kv1.5 in the mechanism of HPV. Not only do mice lacking Kv1.5 have suppressed HPV, but in a different model, CH-PHT, loss of Kv1.5 is associated with blunted HPV which can be restored by Kv1.5 gene transfer. This invention also supports the hypothesis that a K+ channel deficiency state may be involved in the pathogenesis of PHT as described in Yuan X-J, et al., (1996) Circulation 94:1-49, and Archer S, and Rich S, (2000) Circulation 102:2781-91.

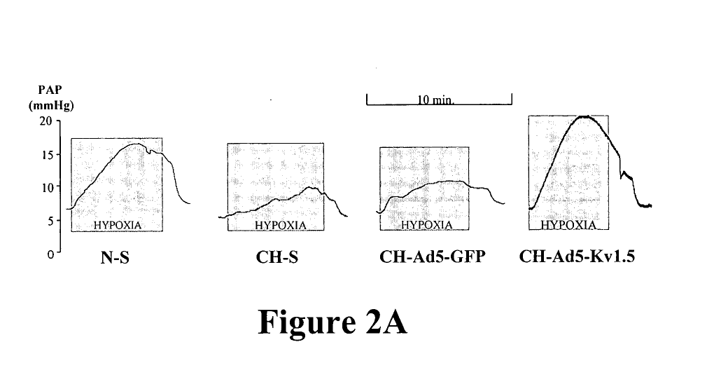

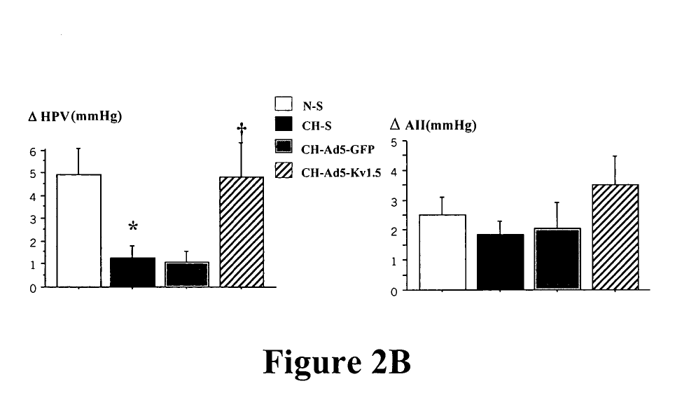

Acute exposure to segmental alveolar hypoxia results in vasoconstriction of small arteries supplying hypoxic alveoli. In the setting of acute, focal airway hypoxia, HPV is an adaptive response that shunts blood from hypoxic alveoli to non-hypoxic alveoli hence optimizing systemic oxygenation. However, it is known that if exposure to alveolar hypoxia continues, particularly if the hypoxia is global, HPV is suppressed. The present invention shows that in rats with confirmed PHT the response to acute hypoxia, but not AII, was depressed, as shown in FIG. 2, consistent with previous reports described in McMurtry I F, et al. (1980) Am J Physiol. 238:H849-57 and Reeve H L, et al., (2001) J Appl Physiol. 90:2249-56. Teleologically, this decrease in HPV may be advantageous in that it minimizes right ventricular strain resulting from global pulmonary vasoconstriction under circumstances in which HPV cannot augment O₂ uptake. Thus, although CH-PHT may be initiated by HPV, this response is downregulated and the PHT appears to be sustained by the remodeling of the pulmonary vasculature, characterized by distal extension of smooth muscle to previously nonmuscular arteries and medial hypertrophy.

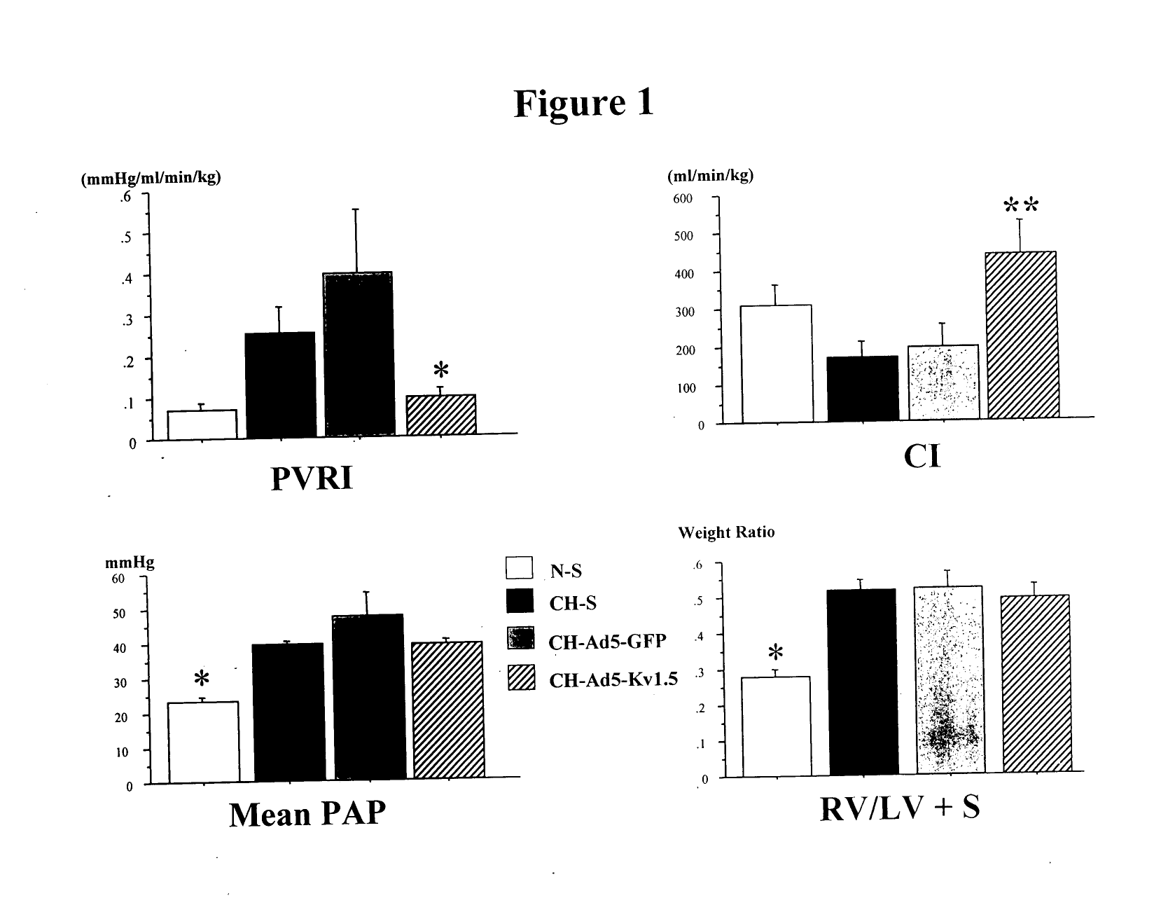

The loss of Kv1.5 expression appears to be important to the pathogenesis of various forms of PHT, including CH-PHT, as described in Reeve H L, et al., (2001) J Appl Physiol. 90:2249-56; Michelakis E D, et al., (2002) Circulation;105:244-50; and Yuan X-J, et al., (1996) Circulation 94:1-49. This concept is now supported by the finding that augmentation of Kv1.5 channel expression by gene transfer reduces PHT, as shown in FIG. 1. The reduction in PVRI was largely the result of increased cardiac index, rather than a fall in mean PAP, as shown in FIG. 1. This type of response is not uncommonly seen in patients with PHT, as described in Rich S, et al. (1985) Am J Cardiol. 55:159-163. Because both the aortic pressure, measured by a high-fidelity catheter, and SVRI were not different between in CH-S versus CH-Ad5-Kv1.5 groups, this suggests that the increased CI is due to decreased RV afterload (pulmonary vasodilatation). The possibility of systemic leak of nebulized Kv1.5 transgene cannot completely be excluded, which theoretically could increase CI by enhancing cardiac inotropy or promoting peripheral vasodilatation. A decrease in RV afterload did not reverse right ventricular hypertrophy. It is likely that regression of RVH requires a longer therapeutic interval than the 3 days allowed post gene transfer in this protocol.

What is the molecular mechanism of the loss of HPV in CH-PHT? There is substantial evidence that Kv channel inhibition initiates HPV. In particular, selective inhibition of Kv1.5, an O₂ and 4-AP-sensitive Kv channel, whether by administration of an antiKv1.5 antibody, as described in Archer S L, et al. (1998) J Clin Invest. 101:2319-30, or by Kv1.5 gene deletion, as discussed in Archer S L, et al., (2001) FASEB J. 15:1801-1803, reduces O₂ sensitive Kv current in isolated PASMCs and also significantly impairs HPV. Kv1.5 is important to HPV and its loss reduces HPV. Kv1.5 expression in PAs is also selectively downregulated in both human pulmonary arterial hypertension, as described in Yuan X J, et al., (1998) Lancet 351:726-7 and experimental models of pulmonary hypertension, as described in Reeve H L, et al., (2001) J Appl Physiol. 90:2249-56 and Yuan X-J, et al., (1993) Am. J. Physiol. 264:L116-L123. Kv1.5 is inhibited by anorexigens, such as dexfenfluramine, which have caused outbreaks of human PHT.

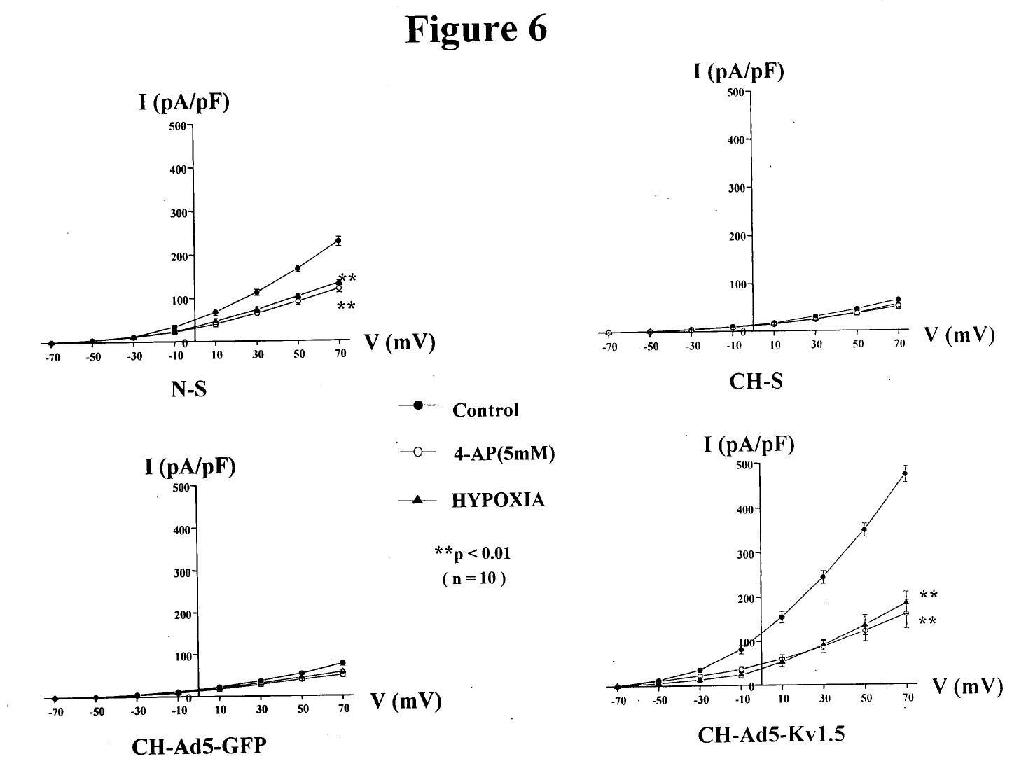

In CH, PASMCs are depolarized, and their I_{K} is less sensitive to 4-AP and hypoxia as shown in FIG. 6. These physiological and electrophysiological abnormalities are associated with, and presumably result from, down regulation of Kv1.5, and Kv2.1.

Kv1.5 expression is dynamically regulated, both at the level of transcription as well as post-translationally. Kv1.5 mRNA has a very short half-life and Kv1.5 protein turnover is also rapid. In addition, the Kv1.5 gene has cyclic adenosine monophosphate and glucocorticoid response elements. The rapid response is consistent with the rapid loss of HPV, that occurs within 1-2 days of CH. CH could decrease Kv1.5 expression by decreasing Kv1.5 gene transcription, destabilizing Kv1.5 mRNA or accelerating turnover of Kv1.5 protein. The fact that CH suppresses genomic, but not episomal Kv1.5 expression, suggests that the control of endogenous Kv1.5 gene is regulated at a transcriptional level, since the human Kv1.5 mRNA and protein are not suppressed by the ongoing hypoxia. It is thought that CH may activate KRE (Kv1.5 repressor element), a dinucleotide repetitive DNA sequence forming a cell-specific silencer, as described in Mori Y, et al., (1995) J Biol Chem. 270:27788-96. When activated, this K+ channel repressor element inhibits transcription of the Kv1.5 gene. The episomal location and promiscuous promoter driving expression of human Kv1.5 in this rat model were not susceptible to downregulation of CH.

As discussed above the present invention also relates to a vector that permits the delivery of an important human Kv channel to the pulmonary circulation. As shown in the examples the use of Kv1.5 cloned from the PA of a normal human allows the detection of only the specific expression of the human gene by qRT-PCR, without interference from endogenous rat Kv1.5. The inclusion of GFP in the vector allows detection of transgene expression using fluorescent microscopy without tissue destruction.

As discussed above, the present invention provides for Kv gene transfer, for example Kv1.5 gene transfer, via airway nebulization in vivo to increase transgene expression in resistant PAs and reverse increased PVRI.

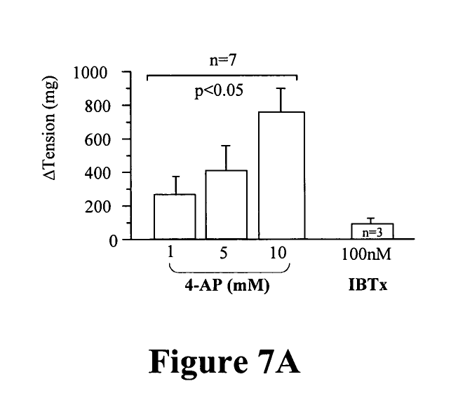

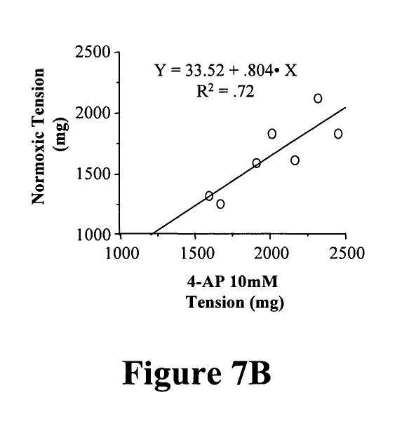

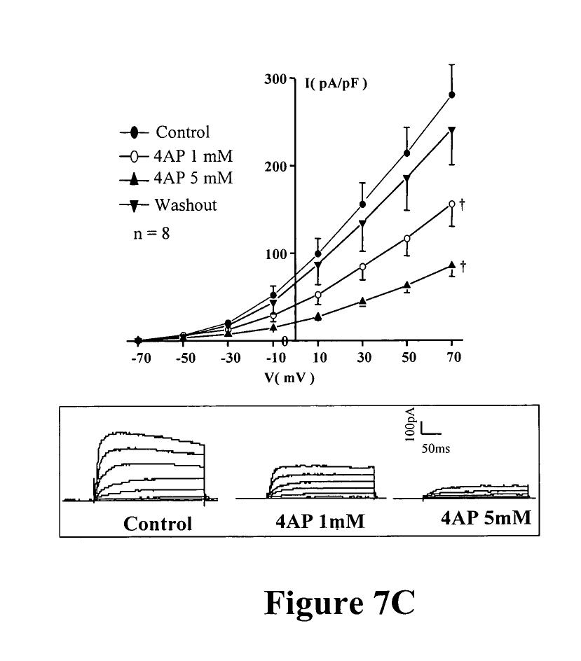

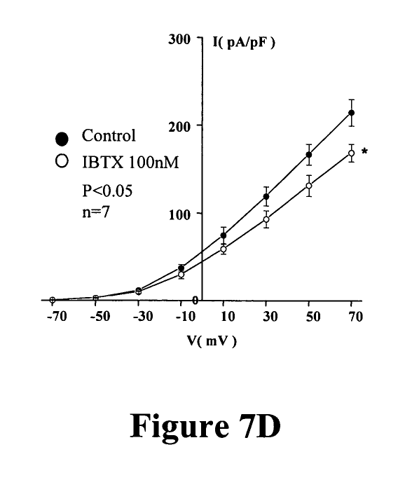

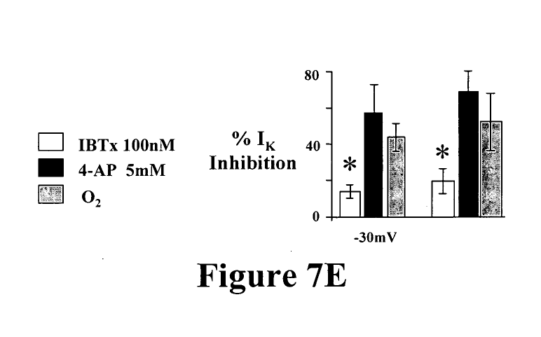

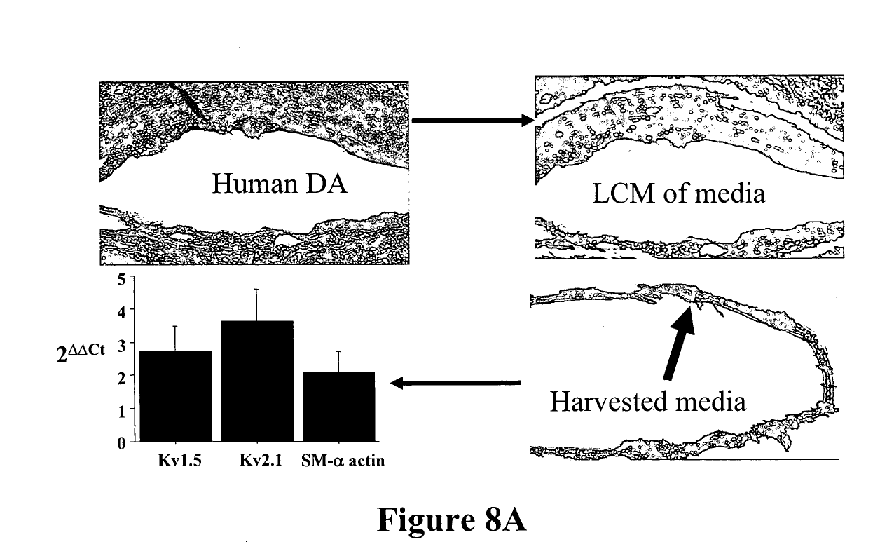

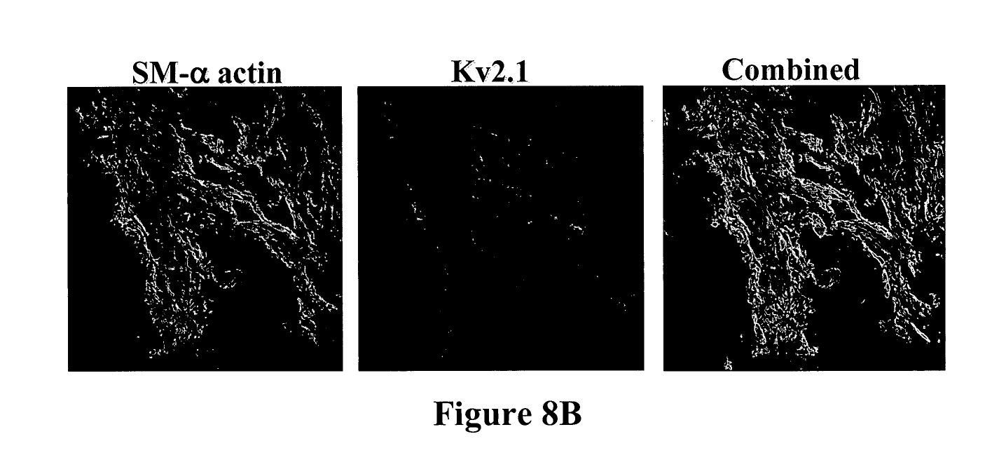

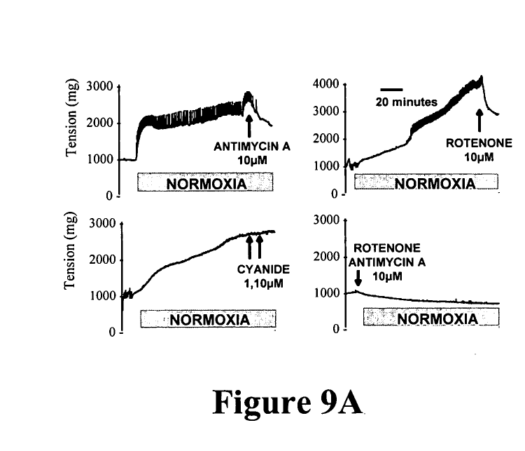

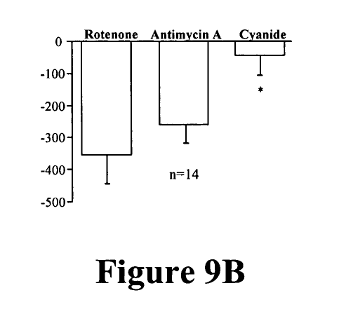

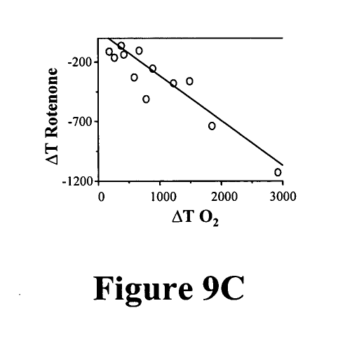

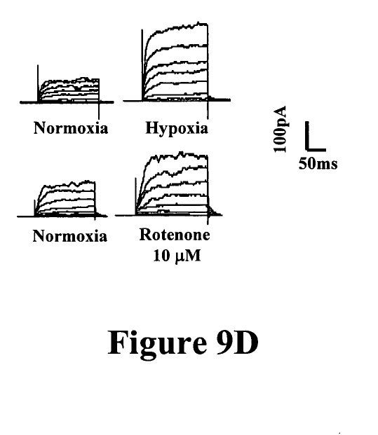

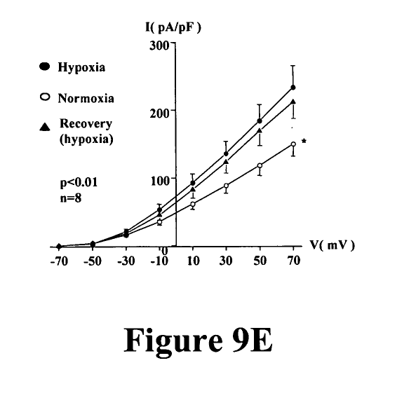

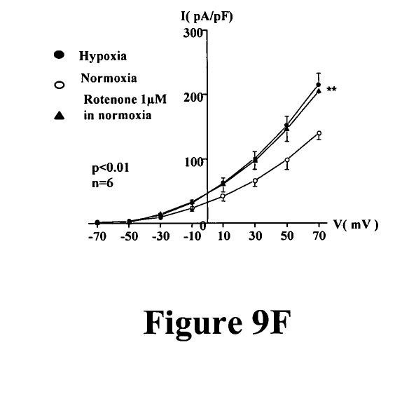

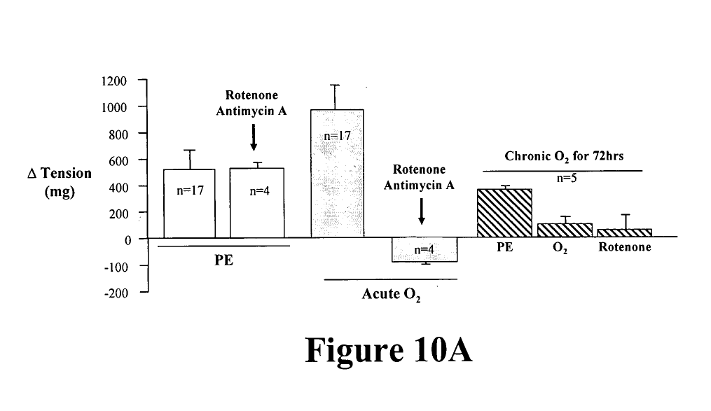

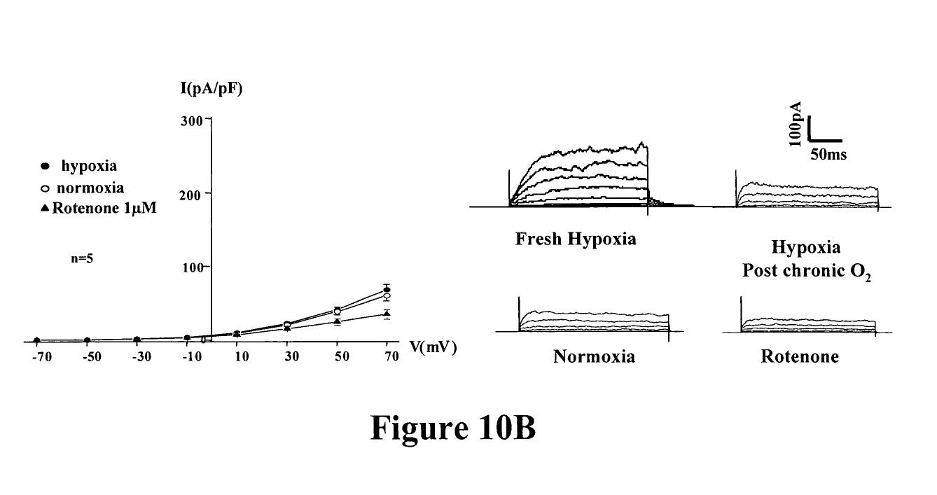

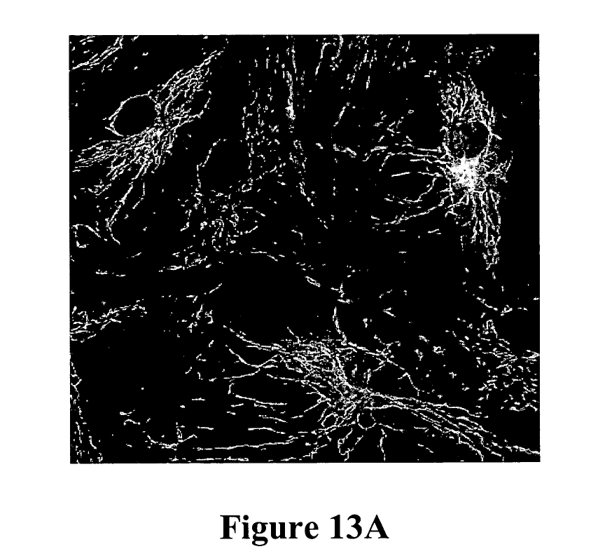

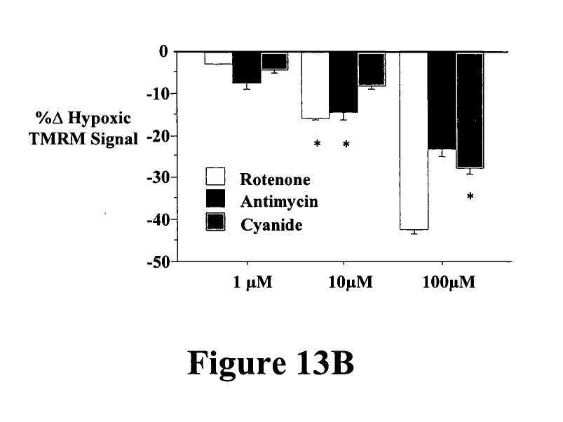

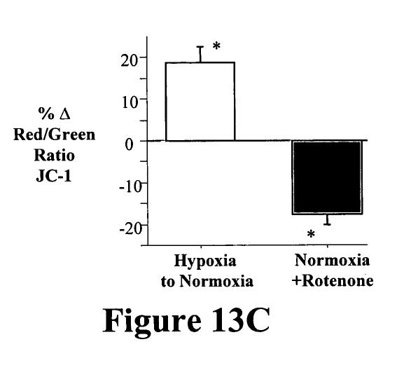

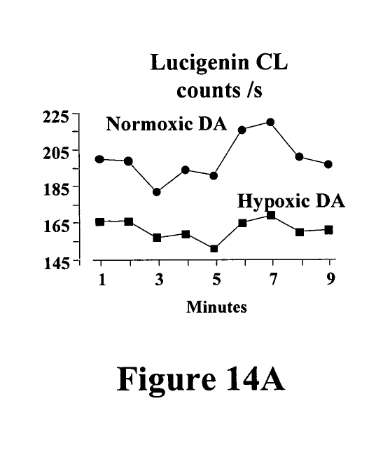

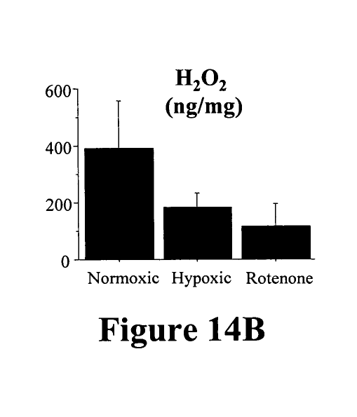

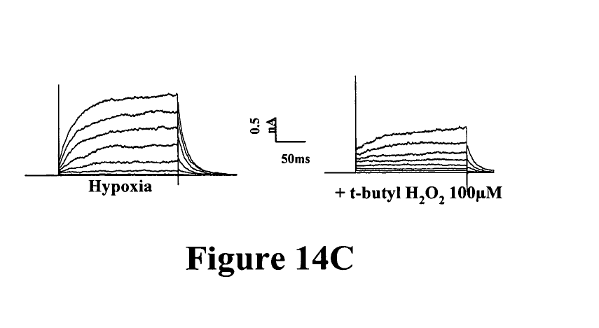

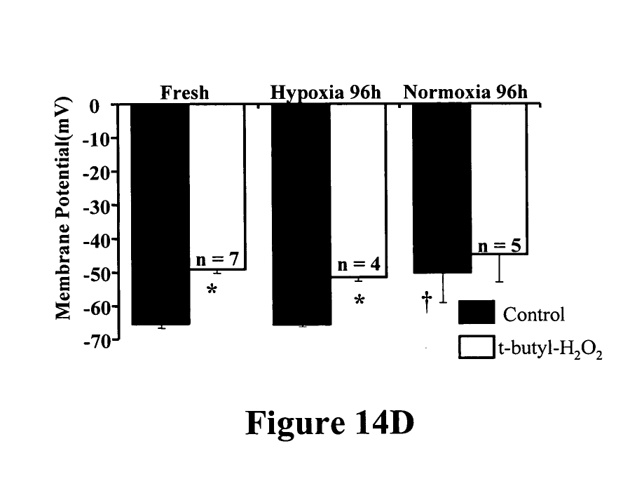

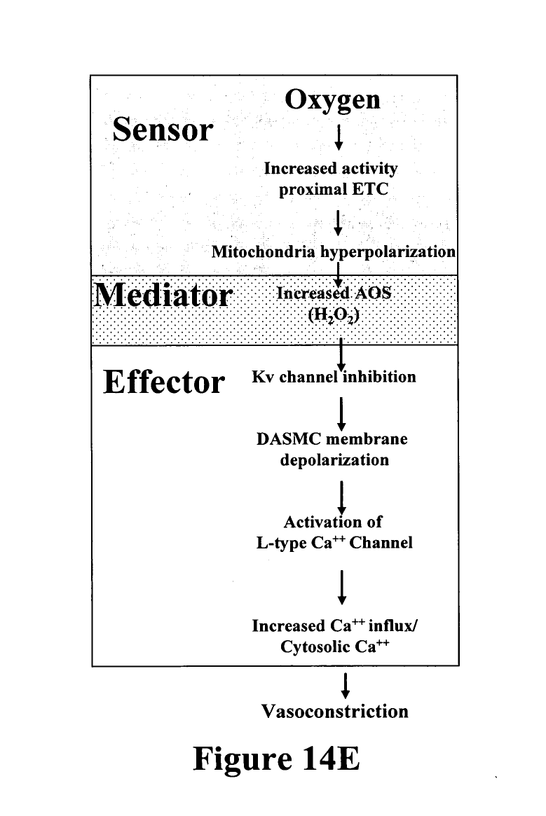

The present invention further illustrates the mechanism of O₂ sensing in the human DA. As can be seen in FIGS. 7 and 8, Kv channels are the effectors of O₂-constriction. Inhibiting these channels, whether by 4-AP or O₂ leads to vasoconstriction. The proximal mitochondrial ETC serves as the O₂ sensor, as shown in FIGS. 9, 10A, and 13. Inhibition of the complex I or III inhibits O₂ constriction. Consistent with data in rabbit DA, the mediator linking the sensor and effector in the human DA appears to be a ROS (H₂O₂) (FIG. 14). The ability of hypoxia and proximal ETC inhibitors to depolarize ψm (FIG. 13) provides a probable mechanism by which hypoxia alters ROS production. In addition, a new model of ionic remodeling is disclosed which is useful in understanding the relative contributions of Kv channels and mitochondria to O₂ -sensing in the human DA (FIGS. 10-12). Together, these findings suggest that ROS produced in the proximal ETC in response to O₂ could be redox mediators that link the mitochondrial sensor to the Kv effector and thus control tone, as illustrated schematically in FIG. 14D.

Roulet and Coburn first demonstrated that O₂-induced DA constriction is associated with membrane depolarization, as described in Roulet M. J., and Coburn R. F. (1981) Circ. Res. 49:997-1002. Although the K⁺ channel effector mechanism is widely conserved amongst O₂ sensitive tissues, the type of K⁺ channel and downstream response to channel inhibition may vary amongst species, between tissues and with maturation. The present invention discloses the mechanism of O₂ constriction in term human DA, maintained in their normal hypoxic environment, except when they were intentionally exposed to acute or chronic normoxia. Despite the congenital heart disease, these DAs have normal responses to O₂ with similar thresholds for onset of O₂ constriction and maximal O₂ responses, as described in Tristani-Firouzi M, et al., (1996) J. Clin. Investigation 98:1959-1965, and Michelakis E, et al., (2000) Lancet 356:134-7.

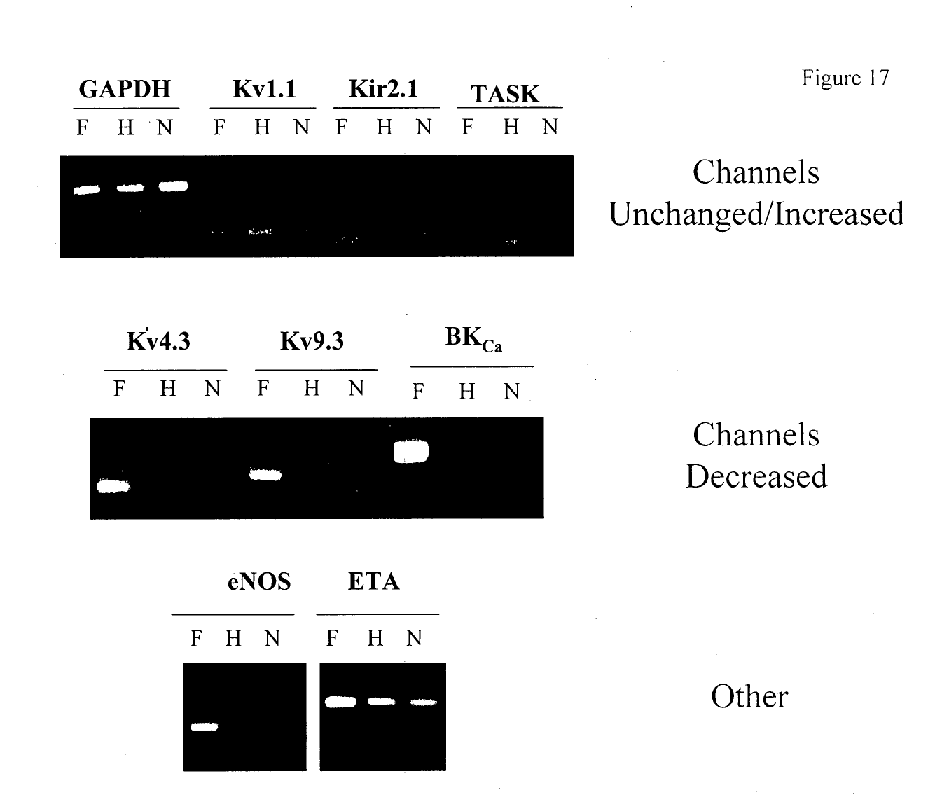

In the human DA, O₂ and 4-AP cause nonadditive vasoconstriction. Nifedipine, an inhibitor of L-type Ca⁺⁺ channels, blocks both 4-AP and O₂ constriction consistent with an obligatory and coordinated role for Kv and Ca⁺⁺ channels in O₂ constriction. The present invention confirms the ability of Kv, but not BK_{Ca}, channel blockers to constrict the human DA and demonstrates a strong correlation between the magnitude of 4-AP constriction and O₂ constriction (FIGS. 7A-B). The basis for these physiological observations is disclosed. The DASMCs express several O₂ and 4-AP sensitive K⁺ channels, which are involved in hypoxic pulmonary vasoconstriction (Kv1.5 and Kv2.1) and O₂ responses in the carotid body, as shown in FIGS. 2 and 17.

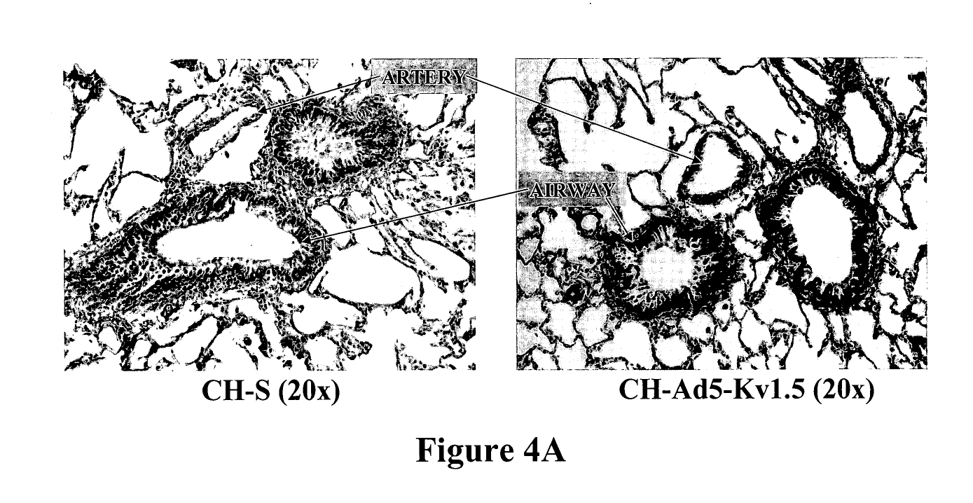

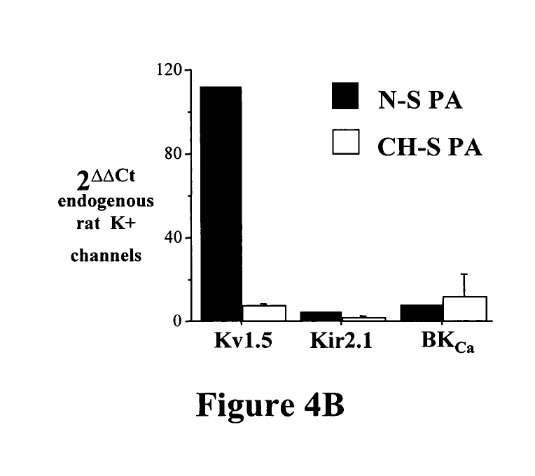

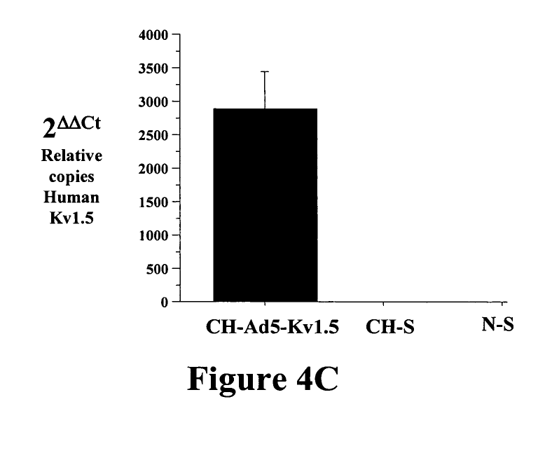

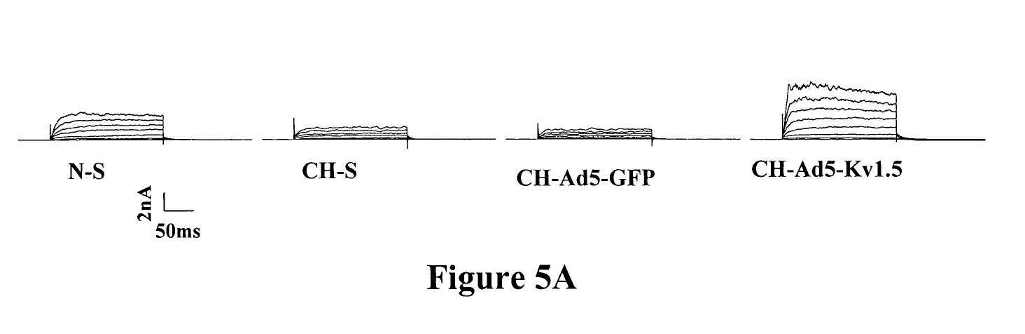

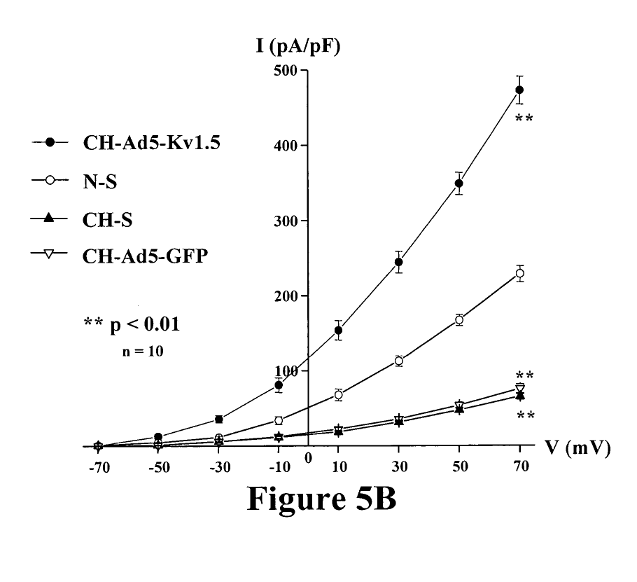

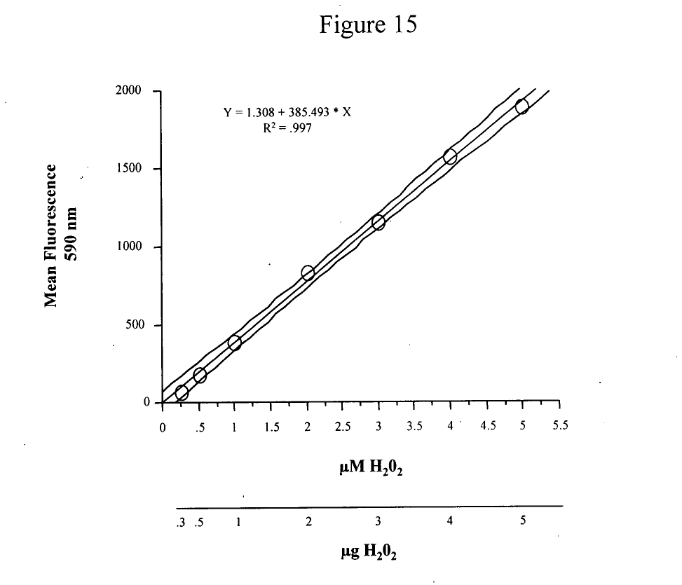

A new model was developed to further understand the role of Kv channels in O₂ constriction. In this model, designed to mimic the conditions in the first days post birth, exposure to tissue culture conditions ex vivo (particularly at normoxic PO₂), inhibited DASMC I_{K}, as shown in FIG. 4B and downregulated several O₂-sensitive Kv channels, including Kv1.5 and Kv2.1, as shown in FIG. 5E. The results shown in FIGS. 15-19 show that expression of other O₂-sensitive channels, Kv4.3, Kv9.3 and BK_{Ca} channels also decrease in this model. However, the lack of effect of iberiotoxin on tension suggests that the role for BK_{Ca} channels may be less important in O₂ response in the DA than in the carotid body. The role of Kv4.3 is somewhat less likely as these channels generate a rapidly inactivating current, unlike the slow-noninactivating O₂ sensitive current in DASMCs.

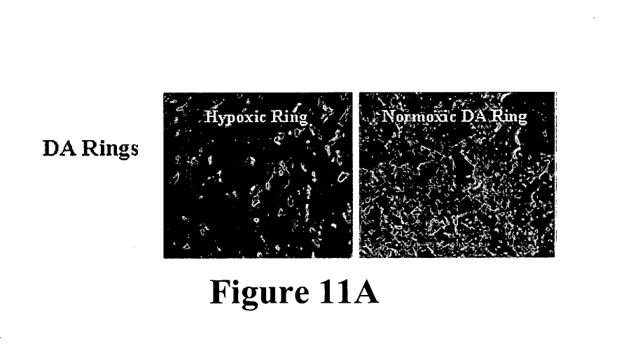

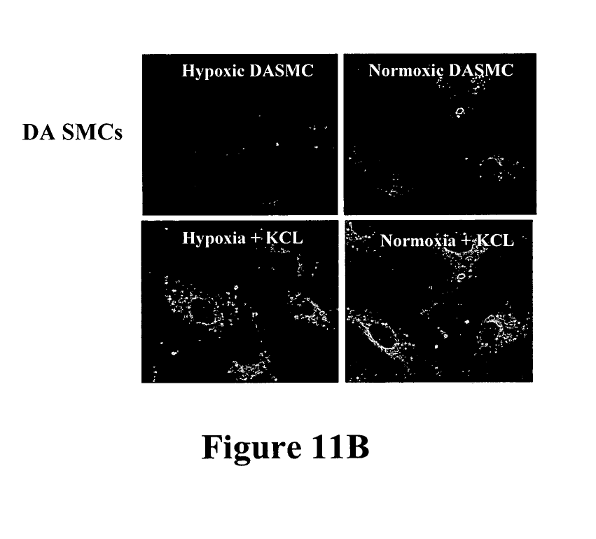

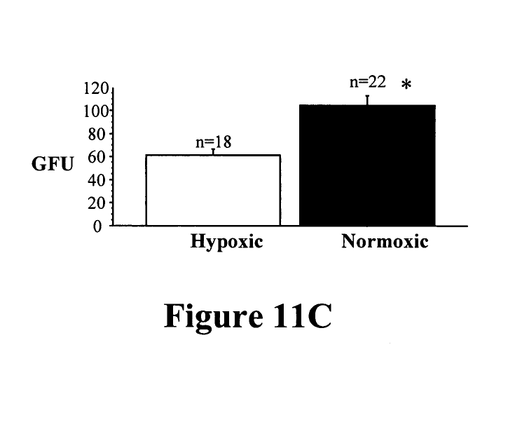

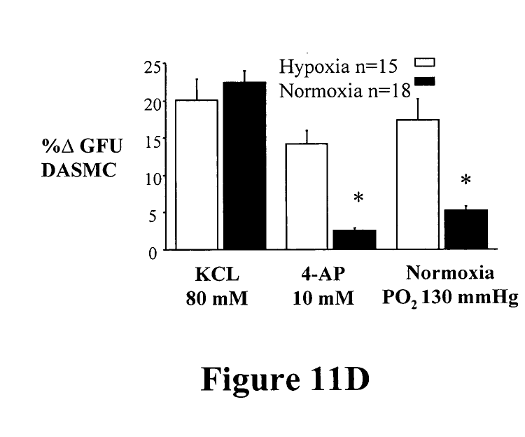

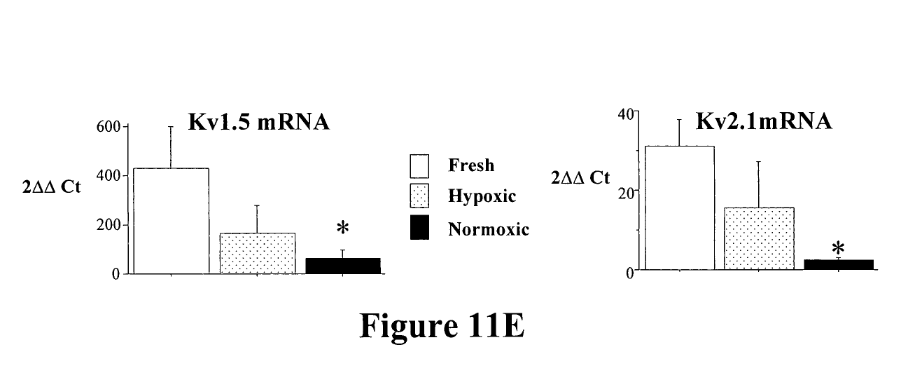

The loss of these Kv channels is associated with membrane depolarization, as shown in FIGS. 11A-C, and impaired ability of the membrane to further depolarize to either O₂ or 4-AP, as shown in FIG. 5D, even hours after the DASMC are returned to a hypoxic environment. These changes in the electrical properties of the DA are termed ionic remodeling. Similar ionic: remodeling and loss of O₂ responsiveness has also been reported in other O₂-sensitive tissues. Loss of acute hypoxic pulmonary vasoconstriction, which occurs with chronic hypoxia, is also associated with downregulation of Kv2.1 and Kv1.5 expression and loss of the O₂-sensitive portion of I_{K}, as described in Reeve H L, et al., (2001) J Appl Physiol. 90:2249-56, and Michelakis E D, et al., (2002) Circulation 105:244-50.

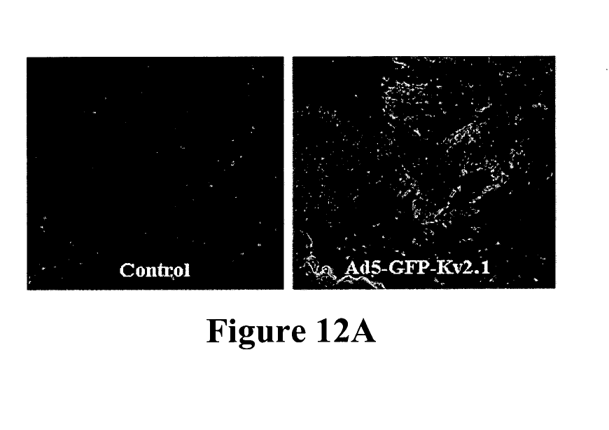

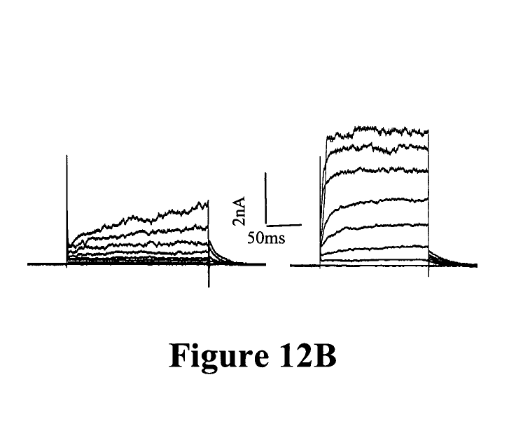

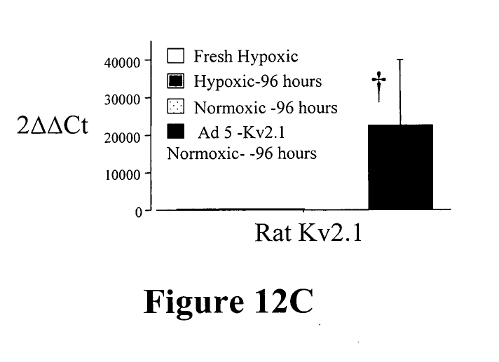

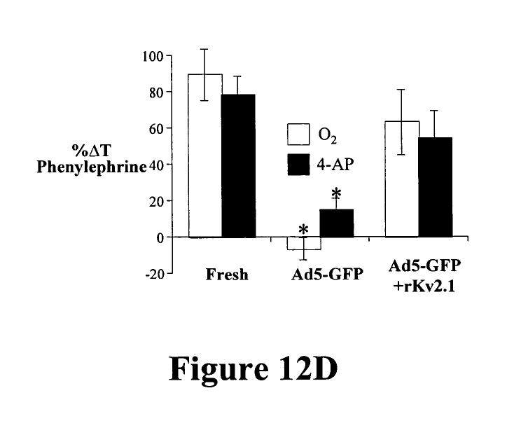

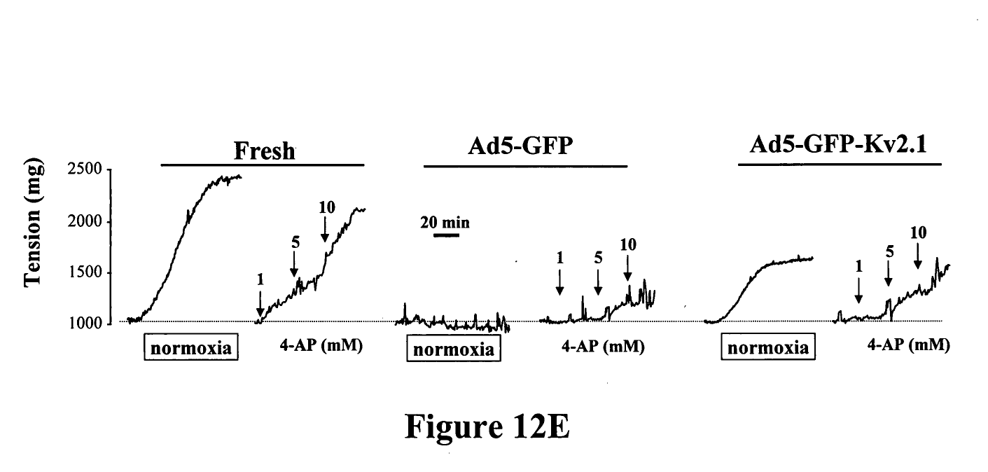

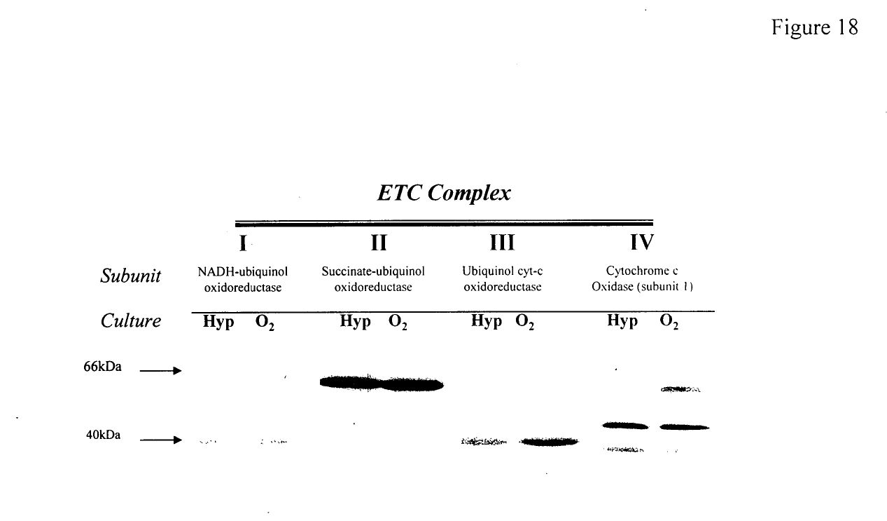

The ionic remodeling model further highlights the importance of the Kv channels to O₂-induced constriction. The reduction in K⁺ current density is associated with loss of sensitivity of current and tone to O₂ and 4-AP (FIG. 10). Furthermore, rotenone's ability to increase I_{K} is lost in this model (FIG. 10B). Coupled with the qRT-PCR evidence for loss of O₂-sensitive channels, but not ETC complexes, as shown in FIG. 18, these data suggest that the loss of O₂ constriction is primarily due to modifications of the Kv effector, rather than the mitochondrial sensor. Consistent with this interpretation, restoration of Kv2.1 expression by ex vivo gene transfer significantly restores the constriction to both 4-AP and O₂ (FIG. 12). The fact that restoration of O₂ and 4-AP constriction is incomplete after Kv2.1 restoration may relate to the fact that other downregulated O₂ and 4-AP sensitive channels (e.g. Kv1.5, Kv9.3, Kv4.3) were not replaced. Alternatively, alterations in function of other components of the contractile apparatus may be abnormal.

Although the pulmonary artery and the DA are contiguous, their response to O₂ is reversed. Hypoxia causes pulmonary vasoconstriction versus DA relaxation. Similar Kv channels are present in both arteries. The fact that the Kv inhibitor 4-AP constricts both arteries suggests that the Kv channels setting E_{M} in the two tissues may be similar. By extension, this implies that their differential O₂ response may relate to differences in either the O₂-sensor or the response of the K⁺ channels to the redox messenger produced by a shared sensor. In human DA, it appears that O₂-sensitive Kv channels are the effectors and the mitochondrial ETC is the O₂ sensor, very similar to the pulmonary artery. What differs is the response to ROS.

The evidence that Kv function and vascular tone are regulated by mitochondria comprises first, rotenone and antimycin mimic hypoxia better than any other class of drugs, suggesting a role for mitochondria in vascular O₂ sensing. Proximal ETC inhibition causes pulmonary vasoconstriction, systemic dilatation, carotid body activation, and DA vasodilatation, as shown in FIG. 9. Rotenone and antimycin, but not cyanide, selectively inhibit O₂ constriction in the human DA and reverse O₂-induced inhibition of I_{K} in DASMCs, as shown in FIGS. 9D-F. The effects of rotenone and antimycin are additive and the combination completely prevents O₂ constriction, without preventing phenylephrine constriction, as shown in FIG. 9A. In addition, the more a DA constricts to O₂ the more relaxation occurs in response to rotenone, but not to cyanide, as shown in FIG. 8C. These findings are a mirror image of those in the pulmonary circulation where ETC inhibitors, also mimicking hypoxia, cause pulmonary vasoconstriction and inhibition of I_{K}. However, in both the pulmonary circulation and DA, hypoxia and ETC inhibitors decrease ROS production, as shown in FIG. 14, suggesting the difference between the vessels relates to their K⁺ channels' response to H₂O₂ and ROS. Indeed, DASMCs depolarize in response to H₂O₂, as shown in FIG. 14, the opposite of the response seen in pulmonary artery SMCs.

The link between the mitochondria, Kv channels and tone is through a redox mediator, rather than ATP levels, as shown in FIG. 14D. Although mitochondria have been dismissed as poor candidates for O₂ sensing because the Km of their cytochromes may be too low for modulation by physiologic levels of hypoxia, they exhibit a reversible inhibition of respiration during prolonged hypoxia due to inhibition of cytochrome c oxidase. Mitochondria are increasingly recognized to be involved in intracellular Ca⁺⁺ control and in redox signaling, in part, because they are important sources of ROS. ψm, a major determinant of cellular redox potential, changes rapidly over a physiological range of PO₂ values in type I carotid body cells, depolarizing in response to hypoxia and metabolic inhibitors. Similarly, hypoxia, rotenone and antimycin depolarize ψm in human DASMCs within 1-2 minutes, as shown in FIG. 13 and this is associated with impaired ROS production, as shown in FIGS. 14 A-B. The confocal images also show that the mitochondria in DASMC, far from being remote from the plasma membrane, form a ubiquitous, filamentous network that permeates the cytosol and is thus positioned to signal changes in PO₂ to all cellular compartments, including the plasma membrane, as shown in FIG. 13.

The examples suggest that the increase in the H₂O₂ levels that occurs with normoxia at the time of birth inhibits DASMC Kv current, causing depolarization and vasoconstriction. Indeed, in human DAs, H₂O₂ production increases as PO₂ rises, as shown in FIGS. 14A-B and H₂O₂ inhibits I_{K} and causing membrane depolarization, as shown in FIGS. 8C-D. Likewise, in rabbit DASMC, intracellular administration of physiological doses of H₂O₂ decreases I_{K} and these effects are inhibited by catalase. The molecular basis for the differential electrophysiological response to H₂O₂ in the pulmonary artery SMC (activation) versus the DASMC (inhibition) is unknown. Possible explanations include tissue-specific differences in K⁺ channel heterotetramer composition, α-subunit splice variants, sulfhydryl redox state of key channel amino acids, or β-subunit expression.

The present invention will now be illustrated by reference to the following examples which set forth particularly advantageous embodiments. However, it should be noted that these embodiments are illustrative and are not to be construed as restricting the invention in any way.

### EXAMPLES

Methods and Materials

The following methods and materials relate to Examples 1-6.

Adenovirus Vector

A recombinant, replication-deficient adenovirus encoding GFP and Kv1.5 was prepared as previously described in Michelakis E D, et al. (2002) Circulation 105:244-50 and Michelakis E D, et al. (2001) Adv Exp Med Biol. 502:401-18.

A 2.1 kb cDNA fragment of Kv1.5 was obtained by reverse transcription of mRNA derived from the proximal pulmonary artery of a cardiac transplant donor. The cDNA was inserted downstream of a CMV promoter in the pAdTrack-CMV shuttle vector. The resultant pAdTrack-CMV Kv1.5 construct was linearized with a PmeI restriction endonuclease digest, transformed together with an adenoviral plasmid (Adeasy-1) into bacterial BJ5183 cells and plated on LB plates containing kanamycin. Subsequent colonies were isolated and cultured, and the resulting plasmid DNA was purified. The plasmid containing Kv1.5 cDNA within the adenoviral DNA was selected, amplified, purified, linearized (Pac I restriction endonuclease), and transfected into HEK 293 cells with LipofectAmine reagent. Plates that demonstrated complete cell lysis were collected and analyzed for the expression of human Kv1.5 using polymerase chain reaction. Multiple rounds of Ad5-Kv1.5 replication were performed in HEK 293 cells. The resulting virus was isolated, precipitated, and concentrated by step-wise discontinuous CsCl gradient. The final viral titer was 1.5×10⁹ pfu/mL.

Exposure to Hypoxia

Rats developed CH-PHT by being placed in a normobaric hypoxic chamber (Reming Bioinstruments, Redfield, N.Y.). They were gradually acclimatized to a final 10% O₂ environment over a 2week time period of increasing hypoxia, as previously described in Michelakis E D, et al. (2002) Circulation 105:244-50.

In Vivo Gene Delivery to the Lung

All rats were housed, 2-3 per cage, and exposed to a 12:12 hour light cycle, and fed standard rat chow and water ad libitum. Adult male, Sprague-Dawley rats were randomized into the following groups: Normoxic-saline (N-S), CH saline (CH-S), CH-adenovirus (5)+green fluorescent protein reporter (CH-Ad5-GFP), and CH+Ad-5+GFP+Kv1.5 gene (CH-Ad5-Kv1.5). The dose of virus was based on preliminary experiments, showing that this dose caused demonstrable Kv1.5 transgene expression without undue toxicity. Each rat was anesthetized with ketamine (60 mg/kg IP) and rompun (10 mg/kg IP, both drugs form Health Sciences Laboratory Animal Services, Edmonton, Alberta) and oro-tracheally intubated with PE-240 tubing (Intramedic, VWR Canlab, Mississauga, ON) during tracheal trans-illumination. The rats breathed spontaneously while intubated. Solution, containing either sterile saline, Ad5-GFP, or Ad5-GFP-Kv1.5 in 100 μL was nebulized via an intratracheal microspray device (Penn-Century Inc. MicroSprayer, Philadelphia, Pa.) placed in the endotracheal tube lumen. The animals were then allowed to recover from anesthesia spontaneously. The CH rats were returned to the hypoxic chamber after recovery (24-48 hours). Experimentation began 72 hours after treatment to allow infection and transgene expression to occur. Sample sizes (n/group) were: hemodynamics (6), isolated lung perfusion (7), immunoblotting, RT-PCR, immunohistochemistry, and confocal imaging (3), and patch clamping (10).

Hemodynamic Measurements

Pulmonary artery pressure (PAP) was measured in closed chest rats with a 1.4F, micromanometer-tipped catheter (Millar Instruments, Houston Tex.), as previously described in Michelakis E D, et al. (1999) J Pharmacol Exp Ther. 291:1143-9. The mean PAP was determined by electronic averaging over one minute. Left ventricular end-diastolic pressure (LVEDP) was measured via retrograde cannulation of the left ventricle and systemic blood pressure (SBP) via the right common carotid artery. Cardiac output was measured using the Fick method, as discussed in Michelakis E D, et al. Circulation. 2002;105:244-50. Cardiac index (CI) was calculated as CO/weight (kg). Pulmonary vascular resistance index (PVRI) was calculated as PAP-LVEDP/CI. As a measure of right ventricular hypertrophy (RVH), the ratio of RV/LV+septum (g) was measured as previously described in Archer S L, et al. (1989) J Appl Physiol. 66:1662-73. Systemic Vascular Resistance Index (SVRI) was measured as MAP-right atrial pressure/CI.

Isolated Lung Perfusion

The isolated perfused lung model was performed as previously described in Archer S, et al. (1987) Cardiovasc Res. 21:928-32. Flow of perfusate (Krebs solution containing 4% bovine serum albumin) was maintained constant at 0.04 mL/g/min by a continuous roller pump. Therefore, changes in PAP solely reflected changes in pulmonary vascular resistance. Throughout the protocol the lungs were ventilated with either hypoxic humidified gas (2.5% O₂, 5% CO₂, balance N₂) or normoxic humidified gas (20% O₂, 5% CO₂, balance N₂). The experimental protocol was performed in the presence of the nitric oxide synthase inhibitor N^{G}-nitro-L-arginine methyl ester (L-NAME, 10⁻⁵ M), and the cyclooxygenase inhibitor meclofenamate (10⁻⁵ M). Angiotensin II (AII, 10⁻⁶M) was given prior to the hypoxic challenge, to allow comparison with a constrictor that does not primarily act via inhibition of O₂ sensitive Kv channels. Cycles of AII-hypoxia-normoxia were repeated 3 times, at which point the magnitude of AII and hypoxic constrictions had plateaued. ΔAII (mmHg) and ΔHPV were calculated during the third cycle, because the magnitude of constriction reaches a plateau after 3 cycles. Blood gas measurements of the perfusate were performed to ensure hypoxia, normoxia and a physiologic pH. Arterial blood gases in normoxia and hypoxia were pH 7.33±0.01, PCO₂31±2.5, PO₂ 134±3.7, and pH 7.37±0.02, PCO₂31±1.6, PO₂42±2.1, respectively. There was no significant difference in pH and PCO₂.

Immunoblotting

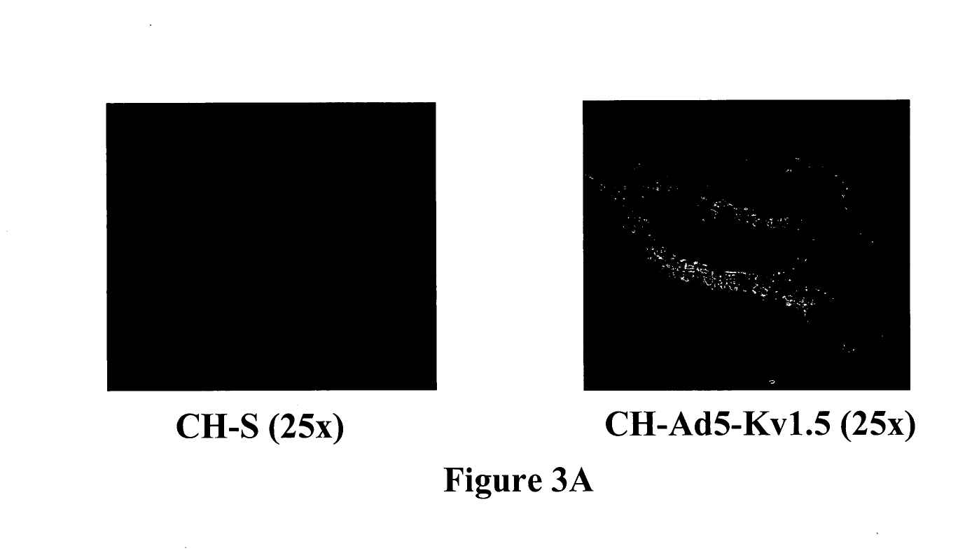

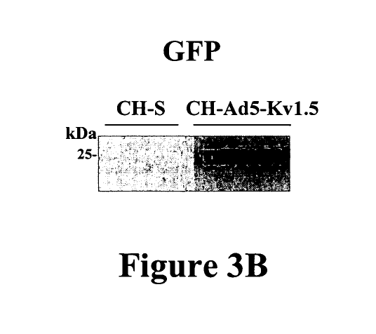

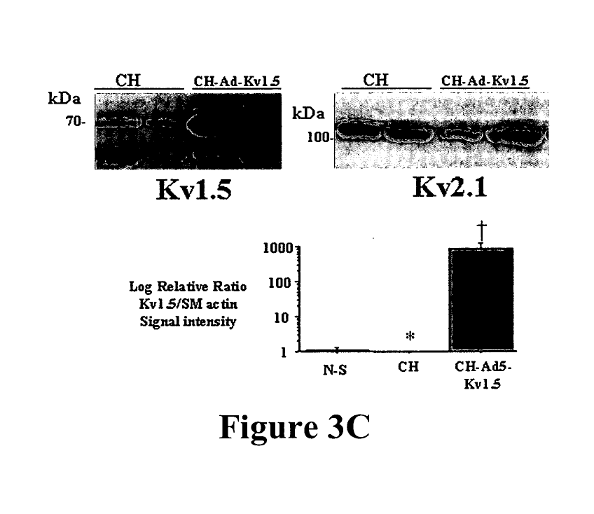

Immunoblots were performed and analyzed as previously described in Michelakis E D, et al. (2002 ) Circulation 105:244-50, using Kv1.5 and Kv2.1 channel (Alomone, Jerusalem, Israel) and GFP antibodies (200 μL diluted 1:1000, Clontech Laboratories, Palo Alto, Calif.) on homogenized lungs. Signal intensity of the immunoreactive Kv bands were normalized to the expression of α-SM actin. The commercially available antibody for Kv1.5 demonstrated cross-reactivity between rat and human Kv1.5, as illustrated in FIG. 3C.

Real-Time Polymerase Chain Reaction

To specifically assess the exogenous human Kv1.5 we used quantitative real-time reverse transcription polymerase chain reaction (qRT-PCR). The primers were designed to be selective for human Kv1.5 and did not detect rodent Kv1.5. Total RNA was extracted using RNEasy Mini Kit (Qiagen, Mississauga, Canada) for a total of 25 μg quantified with UV spectrophotometry. GAPDH was used as a housekeeping gene. The reaction used 25 ng of RNA in 25 μL volume using Kv1.5 primer (500 nM), and a TaqMan probe that we designed (200 nM). Reverse transcription proceeded for 30 min at 48° C., denaturing for 10 min at 95° C., and annealing at 95° C. for 15 sec. Extension occurred for 1 minute at 60° C. over 40 cycles in ABM PRISM 7700 Sequence Detector System (Applied Biosystems, Foster City, Calif.). Copy number was expressed relative to GAPDH, using the N-S group as a calibrator. mRNA, was expressed as 2 ^{Ct} (Ct is cycle time), a measure reflecting the quantitative expression of the Kv mRNA relative to the reporter, glyceraldehyde phosphate dehydrogenase, GAPDH.

Immunohistochemistry

Immunohistochemistry for Kv1.5 was performed on paraffin-embedded, formaldehyde-fixed lungs counterstained with hematoxylin, as previously described in Reeve H L, et al. Alterations in a redox oxygen sensing mechanism in chronic hypoxia. J Appl Physiol. 2001;90:2249-56. Competition assays have previously shown the specificity of the antibody as discussed in Michelakis E D, et al. (2001) Am. J. Physiol. Lung Cell Mol Physiol. 280:L1138-47 and the controls lacking primary antibody showed no staining.

Confocal Microscopy

Fluorescent confocal images were obtained with a Zeiss Axiovert 100M inverted microscope. Green fluorescence was measured, using an LSM-510 confocal microscope (Zeiss, Toronto, Canada), at the following wavelengths (excitation 488 nm, detection 505-530 nm).

Electrophysiology

Fresh PASMC's were isolated from fourth division PA's by enzymatic dispersion and studied using conventional whole-cell patch-clamp technique at room temperature, as previously described in Archer S L, et al. (1998) J. Clin. Invest. 101:2319-30, and Michelakis E D, et al. (2001) Am. J. Physiol. Lung Cell Mol. Physiol. 280:L1138-47. All cells were voltage-clamped at a holding potential of −70 mV. Currents were evoked in stepwise fashion from −70 mV to +70 mV with test pulses of 200 ms. Whole-cell currents were divided by the capacitance of the cell, yielding current density. The pipette solution (intracellular) contained (mmol/L): KCl 134, KH₂PO₄ 1.2, MgCl₂ 1.0, HEPES 5, pH 7.30, Na₂ATP 5 and EGTA 5.

Drugs and Statistics

All drugs were obtained from Sigma-Aldrich Chemical Co (St. Louis. Mo.), unless otherwise stated. Values are expressed as mean±SEM. Intergroup differences were assessed using factorial ANOVA. Post hoc analysis was performed using a Fisher's probable least significant difference test (Statview 5.0, SAS Institute, Cary, N.C.). A p<0.05 was considered statistically significant.

The following methods and materials relate to Examples 7-12:

DA Rings

DAs excised from 26 neonates with hypoplastic left heart syndrome during the Norwood procedure (age 9±2 days, 12 female, arterial PO₂˜40 mmHg) were usedDAs were transported in iced saline, maintained in hypoxia and used within 1 hour. Ring tension was recorded as previously described in Michelakis E, et al. (2000) Lancet 356:134-7.

Optimal passive ring tension was determined to be 1 g, as defined by the maximal constriction to 80 mM KCl. Tension was recorded using a force transducer connected to a MacLab A-D convertor (AD Instruments, Toronto, Canada). Tension was recorded from rings bubbled with 95% N₂ and 5% CO₂ in Krebs' solution, to mimic in utero conditions (PO₂ 40 mmHg, pH 7.4, pCO₂ 40-50 mmHg). Normoxia was created by bubbling with 20%O₂ and 5% CO₂ (balance N₂) resulting in PO₂ 150 mmHg, pH 7.4, and PCO₂ 40 mmHg. Optimal resting tension was previously found to be 1000 mg, as described in Michelakis E, et al. (2000) Lancet 356:134-7. To exclude the contribution of several endothelial-derived vasoactive substances, the Krebs' solution also contained meclofenamate 17 μM to inhibit PGHS and L-N^{G}-nitroarginine-methylester (L-NAME) 10⁻⁵ M to inhibit NOS.

Whole Cell Patch Clamping Technique

Current and voltage clamp technique, SMC isolation and pipette solutions were performed as previously described in Michelakis E D, et al. (2002) Circ Res. 90:1307-15. The response to O₂, ETC inhibitors and the membrane permeable H₂O₂ analog, t-butyl hydrogen peroxide, were compared between DASMCs enzymatically dispersed from fresh DAs versus those maintained in tissue culture for 72 hours (either under normoxic or hypoxic conditions).

Cells were voltage clamped at a holding potential of −70 mV and currents were evoked by 10 mV steps to +50 mV using test pulses of 200 ms duration. Data were recorded and analyzed using pCLAMP 6.02 software (Axon Instruments, Foster City, Calif.). Current density was calculated (pA/pF) and plotted against voltage.

DASMC E_{M}

DASMC E_{M} was measured both using patch clamp technique in current clamp mode and noninvasively with DiBAC₄(3) (20 μM, bis-barbituric acid oxonol), a potentiometric dye that increases green fluorescence upon depolarization. DiBAC₄ loaded cells or intact DA rings were stimulated with multi-photon excitation at 740 nm and emission was measured in the green range, 505-530 nm. DASMCs were studied after culture in normoxic or hypoxic environment for 4-5 days. The acute change in E_{M} that occurred in response to 4-AP (10 mM), KCL (80 mM) or a switch in PO₂ from 40 to 120 mmHg was measured.

Laser Capture Microdissection

LCM utilizes a microscope platform combined with a low energy infrared laser to melt a plastic capture film onto the flash frozen human ductus arteriosus. The PixCell II LCM System (Arcturus Engineering, Mountain View, Calif.) is used to selectively remove specific tissue layers from the DA wall. The machine settings were as follows: slice thickness 7 μM, power 45 mW, duration 1.5 ms. The freshly section ductus was mounted without a coverslip on a DNA-free microscope slide. The tissue was dehydrated using the HistoGene LCM Frozen Section Staining Kit and then the dissected section was placed on a thermoplastic membrane pre-mounted on optically transparent caps. After visual confirmation of the adequacy of the dissection by examining the tissue in the cap, the specimen was placed in RNA-later® for subsequent qRT-PCR.

Real-Time Polymerase Chain Reaction

Quantitative real-time Polymerase Chain Reaction (qRT-PCR) was used to quantify human Kv channel mRNA, relative to GAPDH. Total RNA was extracted using RNeasy Mini Kit (Qiagen, Mississauga, Canada) and quantified with UV spectrophotometry. Quantitative real-time polymerase chain reaction (qRT-PCR) was used to quantify human Kv channel mRNA and expression of rat Kv2.1 mRNA transgene. The TaqMan One-Step RT-PCR Master Mix reagent kit was used (Applied Biosystems, Foster City, Calif.). The reaction used 50 ng RNA in 50 μl using the relevant primer (500 nM), and TaqMan probe (200 nM), as designed by us. The assay was performed using an ABI PRISM 7700 Sequence Detector System (Applied Biosystems). Reverse transcription proceeded for 30 min at 48° C. AmpliTaq Gold activation occurred for 10 min at 95 C. Subsequently, 40 cycles of PCR were performed. Each cycle consisted of 15 seconds of denaturing (at 95° C.) and 1 minute of annealing and extension (at 60° C.). 2 Ct is a ratio of the expression of the Kv channel to glyceraldehyde dehydrogenase (GAPDH).

2 ^{Ct} calculation: 2 ^{Ct} is a conversion factor allowing the amount of Kv mRNA to be expressed in terms of copy number relative to the calibrator (the sample with the least amount of Kv mRNA) and normalised to expression of a housekeeping gene, GAPDH. Ct=threshold cycle for target amplification.

The Ct Kv2.1 was computed for each sample (Equation 1). The largest Ct (indicating the smallest amount of Kv2.1) is defined as the calibrator.

CtKv_{2.1}=CtKv_{2.1}−Ct_{GAPDH} (Equation 1)

Then the relative copy number Ct is calculated for each sample using formula 2

Ct=calibrator−CtKv_{2.1} (Equation 2)

In the case of the sample that is experimentally selected as the calibrator (the lowest expressing sample), Equation 2 yields a value of 0 (subtracting the value from itself). By expressing the Ct as an exponent of 2, the copy number in the calibrator becomes 2⁰=1, allowing easy expression of the larger amounts of Kv mRNA in other specimens relative to this value.

Measurement of ROS by Chemiluminescence

The Lucigenin-enhanced chemiluminescence is sensitive for ROS but does not differentiate amongst the redox species. DA rings were loaded with lucigenin 5 μM and placed in a 2 ml heated cuvette at 37° C. Counts were recorded using a Packard 1900CA Liquid Scintillation Analyzer.

H₂O₂ Assay

H₂O₂, was measured using a specific, fluorometric AmplexRed® assay, as previously described in Michelakis E D, et al. (2002 ) Circ Res. 90:1307-15. An independent, specific measure of the candidate redox mediator, H₂O₂, was made using the one-step, fluorometric Amplex Red (Molecular Probes, Eugene, Oreg.) assay. The assay detects H₂O₂ using 10-acetyl-3,7-dihydroxyphenoxazine (Amplex Red reagent) a stable probe that, in the presence of horseradish peroxidase (HRP), reacts with H₂O₂ with a 1:1 stoichiometry to produce the highly fluorescent substance, resorufin. The assay was calibrated (FIG. 15) and found to be linear over the range of 0.25 to 5.0 μM. Freshly harvested DA rings were placed in a normoxic or a hypoxic solution containing AmplexRed and, after 20 minutes, fluorescence was recorded (excitation 530 nm, emission 590 nm), see FIG. 15.

Mitochondrial Membrane Potential

Mitochondrial membrane potential (Δψm) was measured using two independent and well-validated probes JC-1 and TMRM (tetramethyl rhodamine methyl ester), as previously described in Michelakis E D, et al. (2002) Circ Res. 90:1307-15.

First-passage cultured DASMC were loaded with either JC-1 (1 μM) or TMRM (20 nM) for 30 minutes (37° C.) at 37 C. Confocal images were obtained within 30 minutes using a Zeiss Axiovert 100M inverted microscope equipped with an Apochromat 40×1.2 water-corrected objective. Settings (amplifier gain, pin hole size and filter set) were kept constant between experiments.

JC-1, a cationic fluorescent dye that exhibits potential-dependent mitochondrial accumulation is used extensively to study Δψm. JC-1 fluoresces in the red spectra in hyperpolarized mitochondria (high Δψm) and in the green spectra in depolarized mitochondria (low Δψm). KCl (40 mM) was used to clamp the plasmalemmal membrane potential in some experiments. This did not alter JC-1 fluorescence (not shown), confirming the specificity of this potentiometric dye for the Δψm and not the plasmalemmal E_{M}. TMRM binds the inner and outer aspects of the inner mitochondrial membrane and accumulates in the mitochondria at greater quantities than predicted by the Nernst equation. TMRM fluorescence red in proportion to ψm. A 488 nm Argon laser was used for excitation and the resultant red/green fluorescence was quantified in channels 1 and 2 using LP 560 nm and BP 505-530 nm filters, respectively. 8-bit images were collected with a laser loiter time of 4.48 μs/pixel. Pinholes yielded optical slices of 1 μm. Results are expressed as the ratio of red/green channel fluorescence.

Ex-Vivo Gene Transfer

Adenoviruses (serotype 5, Ad5) carrying genes for rat Kv2.1 and green fluorescent protein (GFP) (GFP, Ad5-GFP- Kv2.1 ) or GFP alone (Ad5-GFP), each under a CMV promoter, were constructed using the Adeasy-1 system as previously described in Michelakis E D, et al. (2002) Circulation 105:244-50.

Adenoviruses (serotype 5) were constructed using the Adeasy-1 system as previously described in He T C, et al., (1998) Proc. Natl. Acad. Sci. U.S.A. 95:2509-14; and Michelakis, E D et al., (2002) Circulation 105:244-50. A recombinant adenovirus encoding GFP and Kv2.1 was prepared using Adeasy-1, an adenoviral backbone vector, which contains adenovirus (serotype 5) genomic DNA with E1 and E3 deleted. A 2609 bp cDNA fragment encoding the open reading frame of the rat Kv2.1 channel was excised from its original pBK-CMV plasmid (kindly provided by Dr. K Takimoto, University of Pittsburgh) using restriction endonucleases Not I and Sal I and ligated into the Not I and Sal I sites of pAdTrack-CMV, which contains a kanamycin resistance gene and two cytomegalovirus (CMV) promoters (one promoter located upstream of GFP and the other upstream of Kv2.1 ). When pAdTrack-CMV and Adeasy-1 are co-transformed into BJ5183 cells, the Kv2.1 gene undergoes homologous recombination with the adenoviral backbone resulting in a plasmid that contains the Kv2.1 and GFP genes. The resultant pAdTrack-CMV Kv2.1 construct was linearised with a Pme I restriction endonuclease digest, transformed together with supercoiled adenoviral vector Adeasy-1 into BJ5183 cells and plated on LB plates containing kanamycin. Subsequent colonies were isolated and the plasmid DNA was purified using plasmid purifying columns (Qiagen). The plasmid containing Kv2.1 cDNA within the adenoviral DNA was selected, amplified, purified, linearised and transfected into HEK 293 cells using LipofectAMINE reagent. Five days after transfection, plates that demonstrated complete cell lysis were collected and analyzed for Kv2.1 cDNA using PCR. Upon confirmation of the Kv2.1 cDNA in the viral genome, multiple rounds of Ad5Kv2.1 replication were performed in HEK 293 cells. The resulting virus carrying GFP and Kv2.1 cDNA was isolated, precipitated and concentrated by discontinuous CsCl gradient. The final viral titer obtained for Ad5-GFP-Kv2.1 was 1.5×10⁹ pfu/ml. 24 hours incubation with recombinant replication deficient adenovirus (Ad5-GFP-Kv2.1) achieved 90% infection rates in Chinese hamster ovary cells (CHO) grown and kept under hypoxia.

Vectors carried the genes for rat Kv2.1 and green fluorescent protein (GFP) or GFP alone, each under a CMV promoter. Twelve hours incubation with recombinant replication deficient adenovirus (Ad5-GFP- Kv2.1) transduced 90% of Chinese hamster ovary cells and generated a functional Kv2.1 current. DA were incubated for 12 hours with either vehicle (normal saline), Ad5-GFP or Ad5-GFP-Kv2.1, the virus was then washed off and the vessels were kept in either a normoxic (PO₂˜120 mmHg) or hypoxic (PO₂˜45 mmHg) incubator for 60 hours (total incubation 72 hours). Successfully infected vessels were selected based on their green fluorescence (excitation 488 nm, detection 505-530 nm). The finding that GFP fluorescence is at a much lower intensity than DiBAC₄ fluorescence allowed measurement of E_{M} in infected rings by increasing the threshold for detection of green emission.

Statistics and Drugs

Values were expressed as the mean±SEM. Intergroup differences were assessed by Student's paired t-test or an ANOVA (factorial or repeated measures) as appropriate. Post hoc analysis used a Fisher's probable least significant difference test (Statview 4.02, Abacus Concepts). A P<0.05 was considered statistically significant. Regression analysis was also performed using Statview.

All drugs were dissolved in saline and were obtained from Sigma-Aldrich (St. Louis, Mo.) unless otherwise stated. Antibodies to ETC complexes were purchased from Molecular Probes (Eugene, Oreg.). The antibodies were directed against NADH ubiquinol oxidoreductases (Complex I, alpha complex, ˜39 kDa), succinate-ubiquinol oxidoreductase (Complex II flavoprotein, ˜70 kDa), ubiquinol-cytochrome c oxidoreductase (Core 2 subunit, Complex III ˜45 kDA) and cytochrome c oxidase (COX chain I, ˜40 kDa). Kv channel antibodies were from Alomone (Jerusalem, Israel, respectively. MnSOD antibody was from Upstate Biotech (New York, Lake Placid, N.Y.).

### Example 1

Hemodynamics

CH increased mean PAP in all hypoxic groups compared to N-S (p<0.01, see FIG. 1). There was no significant difference in mean PAP amongst the CH groups. CI was significantly greater in the CH-Ad5-Kv1.5 group compared to CH-S and CH-Ad5-GFP (p<0.05, FIG. 1). PVRI was increased in the CH-Ad5-GFP and CH-S groups but was restored to normoxic levels in the CH-Ad5-Kv1.5 group (p<0.05, FIG. 1). The RV/LV+septum ratio, an indicator of RVH, was significantly higher in all CH groups compared to N-S (p<0.001, FIG. 1). The SBP for the N-S, CH-S, CH-Ad5-GFP and CH-Ad5-Kv1.5 groups was (mmHg±SEM) 100±8, 94±11, 90±8, and 100±5 (p=NS). The SVRI for the N-S, CH-S, CH-Ad5-GFP and CH-Ad5-Kv1.5 groups was (in mmHg/mL/min) 0.34±0.05, 0.63±0.13, 0.83±0.35, 0.25±0.06 (p=0.15).

### Example 2

Isolated Perfused Lungs

HPV was reduced in the CH-Ad5-GFP and CH-S compared to N-S group. HPV was restored to normal levels in the CH-Ad5-Kv1.5 group (p<0.05, see FIG. 2), but not in the group receiving the control virus (CH-Ad5-GFP). The A-II response was not significantly different amongst groups, illustrated in FIG. 2B, *p<0.05 vs. N-S; † p<0.05 vs CH-S.

### Example 3

Confocal Microscopy

FIGS. 3A-C illustrate a demonstration of effective gene transfer to the distal airways and PA. Confocal microscopy revealed GFP fluorescence in CH-Ad5-Kv1.5 and CH-Ad5-GFP groups. GFP was found in airways, small, resistance pulmonary arteries and veins shown in FIG. 3A. There was no GFP fluorescence in the CH-S group, as shown in FIG. 3A.

### Example 4

Kv1.5 and GFP Expression

Immunoblotting for GFP, shown in FIG. 3B and Kv1.5, shown in FIG. 3C, revealed markedly increased expression of both proteins in the CH-Ad5-Kv1.5 group. In the lower part of panel C the mean data are shown on a logarithmic scale, normalized to smooth muscle α-actin was used as a control for total protein loading, (* p<0.05 differs from N-S). As expected, there was no GFP expression in the N-S or CH-S lungs. Kv1.5 protein expression (expressed as a % of the α-SM actin signal) was reduced in CH-S versus N-S, as shown in FIG. 3C. qRT-PCR, performed on individual, isolated resistance PA, demonstrated that endogenous rat Kv1.5 mRNA expression decreased significantly in response to CH, as shown in FIG. 4B. The loss of Kv1.5 was not nonspecific; expression of several other channels was not depressed (Kir2.1 and the large conductance calcium-sensitive K+ channel, BK_{Ca}), as shown in FIG. 4B. Utilizing a specific primer for exogenous human Kv1.5 in qRT-PCR, human Kv1.5 mRNA was exclusively found in the Ad5-Kv1.5 treated lungs, as shown in FIG. 4C. This group also experienced a 3-log order increase in net Kv1.5 protein expression, as shown in FIG. 3C. Interestingly, overexpression of Kv1.5 was not associated with altered expression of another 4-AP sensitive Kv channel, Kv2.1, as shown in FIG. 3C.

### Example 5

Immunohistochemistry

There was minimal Kv1.5 staining in the small PAs of the CH-S group, as shown in FIG. 4A. Using identical technique, Kv1.5 staining was prominent in the pulmonary arteries, veins and airways of the CH-Ad5-Kv1.5 group, as shown in FIG. 4A. Immunohistochemistry for Kv1.5 (brown signal) in both airway and PAs is increased in the CH-Ad5-Kv1.5 group compared to the CH-S group. qRT-PCR demonstrates that endogenous rat Kv1.5 mRNA expression decreased significantly in response to CH. It is important to note the comparison of relative copy number is only valid within a group (i.e. one cannot compare the absolute expression of Kv1.5, BK_{Ca}, Kir channel) expression. qRT-PCR shows that human Kv1.5 mRNA is only present in isolated PAs from CH-Ad5-Kv1.5 lungs.

### Example 6

Electrophysiology

Hypoxia and 4-AP caused a similar, reversible inhibition of I_{K} in N-S PASMCs. In CH-S PASMCs, current density was significantly decreased and sensitivity to both 4-AP and hypoxia was lost, as shown in FIG. 6. CH-Ad5-Kv1.5 treatment increased the magnitude of the normoxic current density, as shown in FIG. 5 and restored the hypoxia and 4-AP sensitivity of the current, as shown in FIG. 6. There was no effect of the Ad5-GFP on the electrophysiological effects of CH, as shown in FIGS. 5 and 6. Gene transfer resulted in current densities that were more than double those in the noninfected normoxic PASMCs, as shown in FIG. 5. Furthermore, the increase in current density occurred across the range of voltages from −50 to +70 mV.

The following examples relate to the assessment of O₂ constriction of the human DA via inhibition of DASMC Kv channels.

### Example 7

Kv Channels Control Tone

Fresh human DA rings were studied in hypoxia to mimic conditions in utero, except when otherwise specified. The Kv channel inhibitor 4-AP (1-10 mM) significantly constricts the human DA in a dose-dependent manner as shown in FIG. 1A; iberiotoxin (IBTx, 200 nM, a BK_{Ca} blocker) does not. The magnitude of O₂ and 4-AP constriction are strongly correlated as shown in FIG. 7B. Although both 4-AP and IBTx significantly inhibit whole cell K⁺ current (I_{K} ) as shown in FIGS. 7C and D, IBTx causes minimal inhibition of I_{K} at potentials close to the resting E_{M} as shown in FIG. 7E. In contrast, O₂ and 4-AP cause similar, significant decreases in I_{K} at −30 mV, as shown in FIG. 7E.

mRNA for Kv1.5 and Kv2.1 is expressed in the media and SMCs of DA, selectively extracted with Laser Capture Microdissection (LCM), as shown in FIG. 8A. Using LCM, a circumferential ring of media was selectively removed from a human DA. The presence of Kv2.1 and Kv1.5 α-subunit proteins and SM α-actin was confirmed by performing qRT-PCR on the isolated ring of media. The expression of Kv1.5 in the DASMC is further confirmed by immunocolocalization (B) in which the SMCs in the media of an intact human tern DA are stained for α-SM actin (green staining) and Kv1.5 (red). The colocalization results in the combined image showing yellow SMCs. Nuclei are stained with Hoecst 33342. Note the SMCs are separated by a significant amount of unstained myxoid tissue.

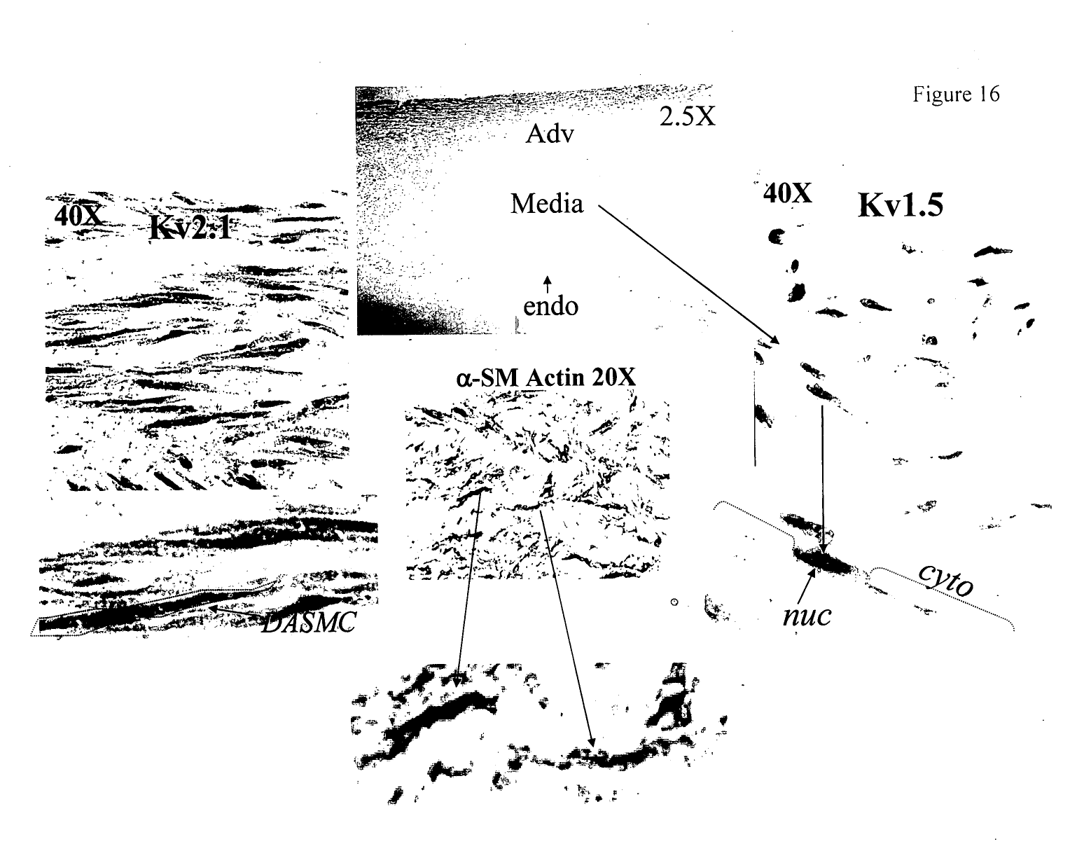

This technique is important because the DA is composed of many cell types and LCM allows specific selection of the media and thus preferential measurement of channel expression in SMCs. Furthermore, immunofluorescent co-localization, shows that Kv2.1, as shown in FIG. 8B and Kv1.5protein is expressed in SM a-actin positive DASMC. This is also confirmed by conventional immunohistochemistry shown in FIG. 16.

### Example 8

The Proximal ETC is the O₂ Sensor.

Both rotenone, a complex I ETC inhibitor, and antimycin, a complex III inhibitor, relax the O₂-preconstricted DA, mimicking hypoxia, as shown in FIGS. 9A-B. In contrast, cyanide does not dilate the DA. Whereas either rotenone or antimycin alone only partially relax the normoxic DA, pretreatment with both inhibitors completely eliminates O₂ constriction, as shown in FIG. 9A, (*P<0.05). ETC inhibitor relaxation is not due to DA damage or nonspecific suppression of tone, since phenylephrine constriction is unaltered by rotenone and antimycin A, as shown in FIG. 9A. Further support for the contention that O₂ and rotenone target the same mechanism is the strong, direct correlation between the magnitude of O₂-constriction and rotenone-relaxation, as shown in FIG. 9C, R²=0.74.

In fresh human DASMC, O₂ rapidly and reversibly inhibits I_{K} as shown in FIGS. 9D-F. Rotenone mimics hypoxia, precisely restoring the current that was acutely suppressed by O₂. In contrast, cyanide (a complex IV blocker, 10 μM) does not alter I_{K}, data not shown. I_{K} is reversibly inhibited by normoxia within 5 minutes, (*, ** P<0.05 values differ from hypoxia and normoxia, respectively).

These data provide strong evidence that the mechanism by which both proximal ETC inhibitors and hypoxia relax the DA is DASMC Kv activation.

### Example 9

A New Model of Ionic Remodeling: DAs in Tissue Culture.

The effects of O₂ and rotenone on tone, as shown in FIG. 10A, K⁺ channel function as shown in FIGS. 10B and 11A-C, and expression as shown in FIG. 11E, were studied in DAs kept in normoxic versus hypoxic tissue culture for 72 hours. The chronically normoxic DA rings retain the ability to constrict to phenylephrine, whereas they lose both the O₂-constriction and the rotenone-relaxation responses, as shown in FIG. 10A. In fresh DA ETC blockers inhibit O₂ constriction (gray bars), without altering the response to phenylephrine (PE, white bars). Chronic exposure of DA rings to O₂, followed by study of ring tension in a hypoxic tissue bath (cross hatched bars) shows that the constrictor response to O₂ (but not PE) is lost and rotenone no longer causes relaxation. SMCs from chronically normoxic DAs have a significantly decreased current density, compared to the freshly isolated cells studied under identical conditions, as shown in FIG. 10B. In addition, when these DASMC are returned to hypoxic conditions, their I_{K} is unresponsive to acute normoxia, 4-AP or rotenone, as shown in FIG. 10B. DASMC dispersed from DAs cultured in chronic normoxia and then studied under hypoxic conditions, have decreased current density. These ionically remodeled cells have lost their responsiveness to both O₂ and rotenone. Representative raw traces are shown to the right of the mean data. The loss of O₂ and ETC sensitivity in chronically normoxic DA is associated with basal, hypoxic E_{M} depolarization and loss of the ability to acutely depolarize in response to either O₂ or 4-AP, whereas the response to KCl is preserved, as shown in FIGS. 11A-D. Confocal microscopy of intact DA rings reveals depolarization (more green) in the half of the ring exposed to O₂ for 72 hours than in the half incubated in hypoxia. The blue stain (Hoechst 33342) marks the nuclei. E_{M} measured in green fluorescent units (GFU, the more depolarized the more green) is more depolarized in chronically normoxic DASMCs compared to chronic hypoxic controls, (* P<0.05). Even when returned acutely to hypoxic conditions, ionically remodeled DASMCs loose their ability to depolarize to 4-AP or O₂ (* P<0.05) but not KCL 80 mM.

The concordant findings of impaired membrane responses to O₂ using both whole-cell current clamp and potentiometric dyes in both DASMC and DA rings excludes the theoretical possibility that this could be an artifact, related to enzymatic dispersion of cells or loss of cell-cell connection.

### Example 10

Ionic Remodeling Results From Downregulation of O₂-Sensitive Kv Channels.

DAs were divided in thirds and both the function and expression of K⁺ channels and ETC complexes were compared in fresh hypoxic DAs, versus DAs cultured in chronic normoxia or chronic hypoxia. In chronic normoxia, mRNA for Kv1.5 and Kv2.1 (measured using qRT-PCR) and Kv4.3, Kv9.3 and BK_{Ca} (measured using conventional RT-PCR, as shown in FIG. 17 where F=Fresh, H=chronic hypoxic culture 96 hours, N=chronic hypoxic culture 96 hours) is downregulated, relative to the housekeeping gene GAPDH, n=5, *p<0.05), as shown in FIG. 11E. There is a statistically insignificant decrease in Kv expression caused by tissue culture in hypoxia.

There is no decrease in Kv1.1, Kir2.1, or TASK expression, measured using conventional RT-PCR, suggesting that the downregulation of these O₂-sensitive channels is somewhat specific, as shown in FIG. 17. Although there was also a trend to lower Kv1.5 and Kv2.1 mRNA in chronic normoxia vs. chronic hypoxia, as shown in FIG. 5E, this was not statistically significant. Expression of selected subunits from ETC complexes I-IV is unaltered by chronic normoxia, as shown in FIG. 18.

### Example 11

Kv2.1 Gene Restoration in the Ionically Remodeled DA.

If the depressed Kv expression in this model is both real and important, it would follow that restoring Kv expression would be sufficient to restore the missing O₂ responsiveness of chronically normoxic DASMCs. Indeed Kv2.1 gene therapy does partially restore O₂ responsiveness, as shown in FIG. 12. DAs were divided into 4 pieces. One piece was studied acutely under hypoxia whereas the others were exposed to normoxia for 12 hours in the presence of vehicle, Ad5-GFP or Ad5-GFP-Kv2.1. This was followed by 60 hours of normoxic incubation to allow gene and protein expression. Of the six human DAs infected, successful gene transfer, as measured by GFP fluorescence, was confirmed in four, as shown in FIGS. 12A-B. The Kv2.1 transgene was derived from rat and, using the species specificity of the qRT-PCR probe, we were able to show that expression of rat Kv2.1 mRNA occurred exclusively in Ad5-GFP-Kv2.1-infected DAs, as shown in FIG. 12C, (n=4). † P<0.01. The transgene yielded functional channels, indicated by the fact that DASMC isolated from Ad5-GFP-Kv2.1 rings had a larger Kv current compared to the DASMC from the noninfected DA, as shown in FIG. 12B. Kv2.1 gene transfer, significantly restores the ability of the normoxic DA to respond to O₂ and 4-AP, as shown in FIGS. 12D and E. In panel D the constrictor responses to O₂ and 4-AP are expressed as a % of the maximal phenylephrine constriction noted in the same rings. Note that infection with a virus containing only GFP is without effect (n=4).

### Example 12

Mitochondria-derived ROS are the redox mediators of normoxic DA constriction. Inhibitors of the proximal ETC and authentic hypoxia rapidly depolarize ψm in DASMCs in primary, hypoxic culture, as shown in FIGS. 13A-D. On the left the DASM mitochondria, shown in FIGS. 13A and B, are imaged with TMRM (nuclei stained with Hoecst 33342) and on the right they are with JC-1 (which shows high ψm in red and depolarized mitochondria in green). Conversely, cyanide 10 μM does not acutely alter ψm, as shown in FIG. 13C. Cyanide also depolarizes ψm, but only at high doses. P<0.05 value differs from control. Rapid increases in PO₂ (from 45 to 100 mmHg) hyperpolarize ψm (increase in the red/green ratio measured using JC-1). Rotenone (10 μM) depolarizes this normoxia ψm (* P<0.05 value differs from control).

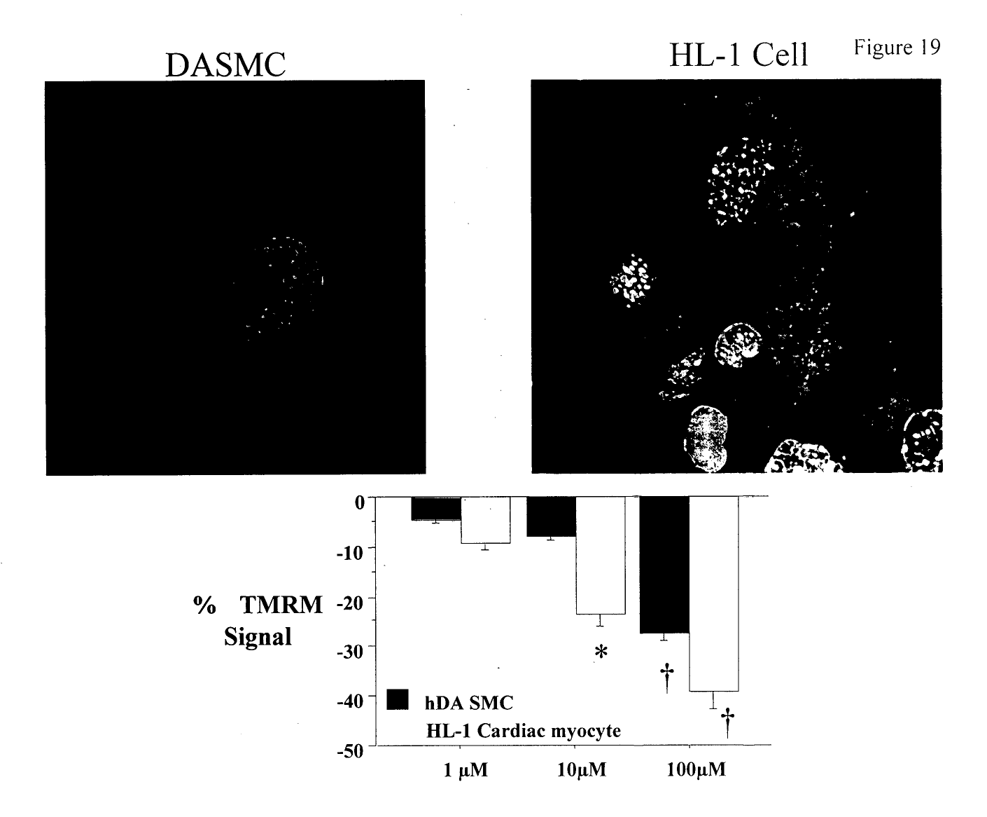

To address the concern that cyanide should (at some dose) be effective in collapsing ψm, its effects on a cardiac HL-1 cell line was also assessed. These experiments showed that 10 μM CN readily depolarizes ψm in HL-1 cells, suggesting diversity in mitochondria between vascular vs. cardiac cells, as shown in FIG. 19. Thus ψm in DASMCs is much more: sensitive to rotenone than to cyanide, consistent with its lack of electrophysiological and hemodynamic effects.

Lucigenin-enhanced chemiluminescence (n=2) and H₂O₂ production (n=5) are increased within minutes by raising PO₂ from 40 to 100 mmHg in freshly isolated, human DA rings, as shown in FIGS. 14A-B. Rotenone decreases H₂O₂ production (FIGS. 14A-B). t-butyl-H₂O₂ mimics the effects of O₂ on DASMC electrophysiology. It inhibits I_{K}, depolarizes fresh hypoxic DASMCs and both these effect are lost after exposure to chronic normoxia, as shown in FIGS. 14C-D. FIG. 14C shows the voltage clamp data illustrating that t-butyl-H₂O₂ inhibits I_{K} in freshly dispersed DASMCs. FIG. 14D shows the current clamp data illustrating that t-butyl-H₂O₂ depolarizes fresh hypoxic DASMCs. This response is preserved in chronically hypoxic DASMCs but is lost in chronically normoxic DASMCs, consistent with their loss of O₂ sensitive Kv channels, (* P<0.05 value differs from control, † P<0.05 from fresh).

The following example relates to a further embodiment of the invention using a replication deficient adenovirus containing a site-specific SM-22 promoter.

### Example 13

A replication deficient adenovirus containing Kv1.5 cDNA driven by site-specific promoter (smooth muscle 22-alpha; SM22a) was created. A polymerase chain reaction (PCR) was performed on cDNA templates (synthesized by reverse transcriptase-PCR from mRNA of a cardiac transplant pulmonary artery donor, in one case, and the ductus arteriosus of a patient with a single ventricle undergoing correction, in another case) using sense and anti-sense oligonucleotides corresponding to the first and last 20-25 bp of the coding region of Kv1.5 respectively. In addition, the anti-sense oligonucleotides ended just 5/ of the stop codon and had the cDNA sequence encoding the 9 amino acid of c-myc insert. Kv1.5myc cDNA was subcloned into the plasmid CR 2.1-TOPO of TA Cloning kit for subsequent propagation. The resulting cDNA from the plasmid was digested with restriction endonuclease EcoRI and was subsequently ligated in a pBluescript plasmid already containing SM22a promoter (a generous gift from Dr. Bloch). The construct carrying SM22a promoter with Kv1.5myc cDNA was removed via restriction digests, purified and ligated into the pShuttle vector. The pShuttle vector contained adenoviral sequence that allows homologous recombination with Adeasy-1. The resultant pShuttle-SM22a-Kv1.5myc construct was linearized with a PmeI restriction endonuclease digest, transformed together with an adenoviral plasmid (pAdeasy-1) into bacterial BJ5183 cells and plated on LB agar containing kanamycin. The selected colonies containing SM22a and Kv1.5myc were isolated, amplified, purified and linearized (PacI endonuclease digest) and transfected into HEK 293 cells with liposome transfection (LipofectAmine reagent). Plates with complete cell lysis were collected and analyzed for Kv1.5 expression using PCR and immunoblotting using anti-Kv1.5 antibody. The Kv1.5myc positive virus was amplified and isolated. The virus was purified by step-wise discontinuous CsCl gradient and the adenovirus was titrated, as previously performed in creating the Ad5-Kv1.5 and Kv2.1 viruses using a CMV promotor as described above.

While the embodiment discussed herein is directed to a particular implementation of the invention, it will be apparent that variations and modifications may be effected thereto, by those of skill in the art, without departing from the scope of the invention.

We claim:

1. An expression vector comprising nucleic acid encoding at least one K+ channel gene operably linked to a promoter for expression in a host cell.

2. A vector according to claim 1, wherein the K+ channel gene is selected from the group consisting of Kv1.5 and Kv2.1/9.3, Kᵥ1.2, Kᵥ3.1, large conductance calcium-sensitive K+ channel genes and BK_{Ca}.

3. A vector according to claim 1, wherein the vector further comprises a reporter gene operably linked to the nucleic acid.

4. A vector according to claim 1, wherein the vector is replication-deficient.

5. A vector according to claim 1, wherein the promoter is a tissue specific promoter.

6. An isolated host cell stably transformed with the expression vector of claim 1.

7. A composition comprising an expression vector comprising nucleic acid encoding at least one K+ channel gene operably linked to a promoter, and a pharmaceutically acceptable excipient.

8. A host cell transformed with nucleic acid encoding at least on K+ channel gene.

9. A method of treating a vascular disease comprising administering to a person having a vascular disease a composition comprising an expression vector encoding at least one K+ channel gene operably linked to a promoter and a pharmaceutically acceptable excipient.

10. A method according to claim 8, wherein the vascular disease is selected from one of pulmonary hypertension and patent ductus arteriosus.

11. A method according to claim 9, wherein the administration is by nebulization.

12. A method according to claim 9, wherein the administration is intravascular.
